(19) United States
(12) Patent Application Publication
(10) Pub. No.: US 2026/0165617 A1
(43) Pub. Date: Jun. 18, 2026

(54) SECONDARY HYDROGEL, POSTGRAFT ENANGLEMENT, AND INTERPENETRATING NETWORKS

(71) Applicant: Senseonics, Incorporated, Germantown, MD (US)

(72) Inventors: Omar Ayyub, Germantown, MD (US); Manav Talreja, Germantown, MD (US); Leopoldo Torres Jr, Germantown, MD (US); Venkata Velvadapu, Germantown, MD (US)

(73) Assignee: Senseonics, Incorporated, Germantown, MD (US)

(21) Appl. No.: 19/419,841

(22) Filed: Dec. 15, 2025

Related U.S. Application Data
(60) Provisional application No. 63/734,420, filed on Dec. 16, 2024.

(51) Int. Cl. A61B 5/1459; A61B 5/145; A61L 31/10

(52) U.S. Cl. CPC A61B 5/1459; A61B 5/14532; A61L 31/10; A61B 2562/12; A61B 2562/16

(57) ABSTRACT

A sensor (e.g., an analyte sensor) that may be implanted partially or fully within a living animal (e.g., a human) and may be used to measure an analyte (e.g., glucose or oxygen) in a medium (e.g., interstitial fluid, blood, or intraperitoneal fluid) within the animal. The sensor may include a housing, a first polymer that includes analyte indicator molecules and covers at least a portion of the housing, and a second polymer that forms an interpenetrating network with the first polymer. The second polymer may prevent biological or protein interference of the sensor.

### CROSS-REFERENCE TO RELATED APPLICATION

The present application claims the benefit of priority to U.S. Provisional Application Ser. No. 63/734,420, filed on Dec. 16, 2024, which is incorporated herein by reference in its entirety.

### BACKGROUND

### Field of Invention

The present invention relates generally to analyte monitoring. More specifically, the present invention relates to a sensor including a housing, a first polymer that includes analyte indicator molecules and covers at a portion of the housing, and a second polymer that forms an interpenetrating network with the first polymer and that reduces absorption and/or adsorption of one or more proteins, one or more macrophages, and/or one or more bacteria to the first polymer.

### Discussion of the Background

A sensor may be implanted (partially or fully) within a living animal (e.g., a human) and used to measure an analyte (e.g., glucose, oxygen, cardiac markers, low-density lipoprotein (LDL), high-density lipoprotein (HDL), or triglycerides) in a medium (e.g., interstitial fluid (ISF), blood, or intraperitoneal fluid) within the living animal. The sensor may include a light source (e.g., a light-emitting diode (LED) or other light emitting element), indicator molecules, and a photodetector (e.g., a photodiode, phototransistor, photoresistor or other photosensitive element). Examples of implantable sensors employing indicator molecules to measure an analyte are described in U.S. Pat. Nos. 5,517,313 and 5,512,246, which are incorporated herein by reference in their entirety.

A sensor may include an analyte indicator, which may be include indicator molecules embedded in a graft (e.g., layer or matrix). For example, in an implantable fluorescence-based glucose sensor, fluorescent indicator molecules may reversibly bind glucose and, when irradiated with excitation light (e.g., light having a wavelength of approximately 378 nm), each indicator molecule may emit an amount of light (e.g., light in the range of 400 to 500 nm) that depends on whether glucose is bound to the indicator molecule.

If a sensor or other medical device is implanted in the body of a living animal, the animal's immune system may begin to attack the sensor or medical device. For instance, if a sensor or other medical device is implanted in a human, white blood cells may attack the sensor or other medical device as a foreign body, and, in the initial immune system onslaught, neutrophils may be the primary white blood cells attacking the sensor. The defense mechanism of neutrophils includes the release of highly caustic substances known as reactive oxygen species. The reactive oxygen species include, for example, hydrogen peroxide.

Hydrogen peroxide and other reactive species such as reactive oxygen and nitrogen species may degrade the indicator molecules of an analyte indicator. For instance, in indicator molecules having a boronate group, hydrogen peroxide may degrade the indicator molecules by oxidizing the boronate group, thus disabling the ability of the indicator molecule to bind glucose. In addition, such reactive species degrade ester-containing polymers of an analyte indicator, for example, as described by Reid, B. et al. PEG hydrogel degradation and the role of the surrounding tissue environment. J. of Tissue Engr. and Regen. Med. 2015.

There is presently a need in the art for improvements in reducing analyte indicator degradation. There is also a need in the art for continuous analyte sensors having increased longevity.

### SUMMARY

The present invention overcomes the disadvantages of prior systems by providing, among other advantages, reduced protein absorption, reduced protein adsorption, and/or reduced analyte indicator degradation.

One aspect of the present invention may provide a sensor including a housing, a first polymer, and a second polymer. The first polymer may include analyte indicator molecules. The second polymer may form an interpenetrating network with the first polymer. In some aspects, the first polymer may cover at least a portion of the housing. In some aspects, the second polymer may reduce absorption and/or adsorption of one or more proteins, one or more macrophages, and/or one or more bacteria to the first polymer.

In some aspects the sensor may include one or more light sources and one or more photodetectors within the housing.

In some aspects, the second polymer may be physically entangled with the first polymer. In some aspects, the second polymer may include analyte indicator molecules. In some alternative aspects, the second polymer may not include analyte indicator molecules.

In some aspects, the sensor may include a plurality of reactive oxygen species (ROS) scavenger molecules covalently linked to the first polymer. In some aspects, the sensor may include a plurality of reactive oxygen species (ROS) scavenger molecules covalently linked to the second polymer.

In some aspects, the second polymer may prevent optical interference of the sensor In some aspects, the second polymer may reduce contact of degradative species with the first polymer. In some aspects, the degradative species may be hydrogen peroxide, a reactive oxygen species, a reactive nitrogen species, enzymes, free radicals, or metal ions. In some aspects, the second polymer may not leach out of or dissociate from the sensor. In some aspects, the second polymer may reduce, inhibit, or prevent electrostatic interactions of the first polymer with one or more proteins.

In some aspects, the first polymer may include co-monomers of four monomers according to Formula Ia: ABCD [Formula Ia]. In some aspects, A may be an analyte indicator monomer. In some aspects, B may be a methacrylate monomer. In some aspects, C may be a polyethylene glycol monomer. In some aspects, D may be a compound or monomer including boronate or boronic acid containing moieties. In some aspects, A may be 0.01 to 10 % by weight of Formula Ia. In some aspects, B may be 1 to 99 % by weight of Formula Ia. In some aspects, C may be 1 to 99 % by weight of Formula Ia. In some aspects, D may be 0.01 to 99% by weight of Formula Ia.

In some aspects, the second polymer may include co-monomers of three monomers according to Formula Ib: EFG [Formula Ib]. In some aspects, E may be a methacrylate monomer. In some aspects, F may be a polyethylene glycol monomer. In some aspects, G may be a compound or monomer including boronate or boronic acid containing moieties.

In some aspects, the co-monomers of the second polymer may be covalently linked via free radical polymerization, click chemistry or step growth polymerization. In some aspects, the F monomers of the second polymer may be thiolene polymerized. In some aspects, the F monomers of the second polymer may include N-hydroxysuccinimide (NHS), dibenzylcyclooctyne (DBCO), amine, epoxide, vinylsulfone, malemide, norebornene, thiol, azide, or alkyne groups. In some aspects, E may be 1 to 99 % by weight of Formula Ib. In some aspects, F may be 1 to 99 % by weight of Formula Ib. In some aspects, G may be 0.01 to 99% by weight of Formula Ib.

In some aspects, the analyte indicator molecules of the first polymer may emit emission light in response to being irradiated by excitation light. In some aspects, the amount of the emission light may vary in accordance with an amount or concentration of analyte in proximity to the first polymer. In some aspects, the second polymer may affect neither the amount of the emission light nor the ability of the analyte to reach the first polymer.

In some aspects, the second polymer may be linked to the first polymer. In some aspects, the second polymer may be linked to the surface of the first polymer. In some aspects, the second polymer may be linked to the first polymer other than by the surface of the first polymer. In some aspects, the linkage between the first polymer and the second polymer may be chemical. In some aspects, the linkage between the first polymer and the second polymer may be physical. In some aspects, the second polymer may be grown from the first polymer. In some aspects, the second polymer may be formed independently of the first polymer. In some aspects, the first polymer and the second polymer may be formed simultaneously. In some aspects, the second polymer may include single linear polymer chains. In some aspects, the second polymer may include cross-linked portions of polymer chains. In some aspects, the second polymer may reduce chemical degradation and/or oxidation of the analyte indicator molecules of the first polymer.

Another aspect of the present invention may provide a method of fabricating a sensor. The method may include applying a first polymer to a housing of the sensor such that the applied first polymer may cover at least a portion of the housing. The first polymer may include analyte indicator molecules. The method may include swelling the first polymer. The method may include soaking the first polymer in a solution of initiator molecules to form a soaked first polymer. The method may include placing the soaked first polymer into a monomer solution to form a second polymer that may form an interpenetrating network with the first polymer. In some aspects, the second polymer may reduce absorption and/or adsorption of one or more proteins, one or more macrophages, and/or one or more bacteria to the first polymer.

In some aspects, the initiator molecules may be selected from a group including free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amine, peroxide/ascorbic acid, ketone/amine), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

In some aspects, the second polymer may be physically linked to the first polymer. In some aspects, a solvent may be used to control the extent to which the second polymer can be physically linked to the first polymer.

In some aspects, the duration that the first polymer is swelled may be used to control the extent to which the second polymer may be physically linked to the first polymer. In some aspects, the second polymer may be physically entangled with the first polymer. In some aspects, the second polymer may be grown from the first polymer.

In some aspects, the second polymer may include analyte indicator molecules. In some aspects, the second polymer may not include analyte indicator molecules. In some aspects, a plurality of reactive oxygen species (ROS) scavenger molecules may be covalently linked to the first polymer. In some aspects, a plurality of reactive oxygen species (ROS) scavenger molecules may be covalently linked to the second polymer.

In some aspects, the second polymer may prevent optical interference of the sensor In some aspects, the second polymer may reduce contact of degradative species with the first polymer.

In some aspects, the degradative species may be hydrogen peroxide, a reactive oxygen species, a reactive nitrogen species, enzymes, free radicals, or metal ions. In some aspects, the second polymer may not leach out of or dissociate from the sensor. In some aspects, the second polymer may reduce, inhibit, or prevents electrostatic interactions of the first polymer with one or more proteins.

In some aspects, the first polymer may include co-monomers of four monomers according to Formula Ia: ABCD [Formula Ia]. In some aspects, A may be an analyte indicator monomer, B may be a methacrylate monomer, C may be a polyethylene glycol monomer, and D may be a compound or monomer including boronate or boronic acid containing moieties. In some aspects, A may be 0.01 to 10 % by weight of Formula 1a. In some aspects, B may be 1 to 99 % by weight of Formula 1a. In some aspects, C may be 1 to 99 % by weight of Formula 1a. In some aspects, D may be 0.01 to 99% by weight of Formula Ia.

In some aspects, the second polymer may include co-monomers of three monomers according to Formula Ib: EFG [Formula Ib]. In some aspects, E may be a methacrylate monomer, F may be a polyethylene glycol monomer, G may be a compound or monomer including boronate or boronic acid containing moieties.

In some aspects, the co-monomers of the second polymer may be covalently linked via free radical polymerization, click chemistry or step growth polymerization. In some aspects, the F monomers of the second polymer may be thiolene polymerized. In some aspects, the F monomers of the second polymer may include malemide, norbornene, thiol, azide, or alkyne groups. In some aspects, E may be 1 to 99 % by weight of formula 1 b. In some aspects, F may be 1 to 99 % by weight of Formula 1 b. In some aspects, G may be 0.01 to 99% by weight of Formula Ib.

In some aspects, the first polymer may emit emission light in response to being irradiated by excitation light. In some aspects, the amount of the emission light may vary in accordance with an amount or concentration of analyte in proximity to the first polymer. In some aspects, the second polymer may affect neither the amount of the emission light nor an ability of the analyte to reach the first polymer.

In some aspects, the method may further include the step of drying the soaked first polymer before placing the soaked first polymer into the monomer solution to form the second polymer. In some aspects, the monomer solution may include a mixture of initiator molecules and monomer molecules. In some aspects, the second polymer may reduce chemical degradation and/or oxidation of the analyte indicator molecules of the first polymer

Another aspect of the present invention may provide a method of fabricating a sensor. The method may include immersing a housing in a solution including a first monomer, a second monomer, and initiator molecules. The method may include forming first and second polymers that at least partially cover the housing. The first polymer may include analyte indicator molecules. In some aspects, the second polymer may form an interpenetrating network with the first polymer. In some aspects, the second polymer may reduce absorption and/or adsorption of one or more proteins, one or more macrophages, and/or one or more bacteria to the first polymer.

In some aspects, the initiator molecules may be selected from a group including free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amines, peroxide/ascorbic acid, ketone/amines), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

In some aspects, the first polymer and the second polymer may be grown simultaneously. In some aspects, the first polymer and the second polymer may be formed at different rates.

In some aspects, the invention may provide a sensor including a housing, a first polymer, and a second polymer. The first polymer may include analyte indicator molecules. The second polymer may form an interpenetrating network with the first polymer. In some aspects, the first polymer may cover at least a portion of the housing. In some aspects, the second polymer may reduce chemical degradation and/or oxidation of the analyte indicator molecules.

In some aspects, the invention may provide a sensor including a housing, a first polymer, and a second polymer. The first polymer may include analyte indicator molecules. The second polymer may form an interpenetrating network with the first polymer. In some aspects, the first polymer may cover at least a portion of the housing. In some aspects, the second polymer may reduce chemical degradation and/or oxidation of the analyte indicator molecules and may reduce absorption and/or adsorption of one or more proteins, one or more macrophages, and/or one or more bacteria to the first polymer.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a schematic view illustrating a system embodying aspects of the present invention.

FIG. 2 is a schematic view illustrating a system embodying aspects of the present invention.

FIGS. 3A-3E illustrate a sensor of the system according to some aspects. FIG. 3A is an exploded view of the sensor of the system according to some aspects. FIG. 3B shows a power source and a coupler of the sensor according to some aspects. FIG. 3C shows circuitry at least partially within a housing of the sensor according to some aspects. FIGS. 3D and 3E show a portion of the housing extending into the coupler of the sensor according to some aspects.

FIG. 4 shows growth of a second polymer from a first polymer of a sensor to form analyte indicator material of a sensor of the according to some aspects.

FIG. 5 shows a sensor of the system including a second polymer grown from a network of a first polymer in a monomer solution utilizing initiator molecules according to some aspects. FIG. 5 includes inset detail showing the second polymer physically entangled with the first polymer according to some aspects.

FIGS. 6A-6C show the first polymer as a polymer network, the second polymer as a polymer network, an interpenetrating network including the first and second polymers, respectively, according to some aspects.

FIGS. 7A-7C show the first polymer, the second polymer, and an interpenetrating network including the first and second polymers according to some aspects.

FIG. 8 is a flow chart illustrating a process according to some aspects.

FIG. 9 is a flow chart illustrating a process according to some aspects.

FIG. 10 is a chemistry diagram showing the 4-Arm PEG-Maleimide (MW=2K), the 4-Arm PEG-Thiol (MW=2K), and the thiol-ene Michael addition with maleimide.

FIG. 11 shows the PEG-Thiol/PEG-Maleimide IPN synthesis process.

FIGS. 12A-12D show the sensor transmission data in 40 mg/mL Bovine Serum Albumin (BSA). FIG. 12A shows data for the UV reference. FIG. 12B shows data for the glucose signal. FIG. 12C shows data for the blue reference. FIG. 12D shows data for the YOI signal. IPN 10% is shown in red. IPN 7% is shown in blue. A control (reference) is shown with a thick black line.

FIGS. 13A-13D show the sensor transmission data in 10% hemolyzed red blood cells. FIG. 13A shows data for the UV reference. FIG. 13B shows data for the glucose signal. FIG. 13C shows data for the blue reference. FIG. 13D shows data for the YOI signal. IPN 10% is shown in red. IPN 7% is shown in blue. A control (reference) is shown with a thick black line.

FIGS. 14A-14H show the SFT parameters for the sensor post-graft. SFT parameters are shown for the 10% IPN (left), 7% IPN (middle) and reference (right). Data for Kd37_glu is shown in FIG. 14A. Data for S0_glu is shown in FIG. 14B. Data for Smax_glu is shown in FIG. 14C. Data for Mods_glu is shown in FIG. 14D. Data for perMod_glu is shown in FIG. 14E. Data for R0 is shown in FIG. 14G). Data for I0 is shown in FIG. 14H.

FIGS. 15A-15B shows an exemplary interpenetrating network. FIG. 15A shows the primary, polymer-rich HEMA-PEGDA phase. FIG. 15B shows the secondary, hydrogel-rich PEG network phase.

### DETAILED DESCRIPTION

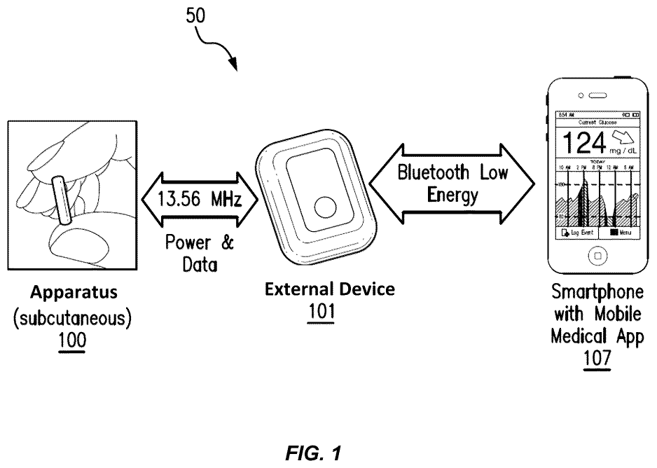

FIG. 1 is a schematic view of an exemplary system 50 embodying aspects of the present invention. In some aspects, the system 50 may be an analyte monitoring system. In some aspects, the system 50 may be a continuous analyte monitoring system (e.g., a continuous glucose monitoring system). In some aspects, the system 50 may include a sensor 100, an external device 101, and/or a display device 107.

In some aspects, the sensor 100 may be an implantable device. In some aspects, the sensor 100 may be a wireless implantable device. In some aspects, the sensor 100 may be a specific sensor (e.g., an analyte sensor). In some aspects, the sensor 100 may include one or more optical sensors (e.g., one or more fluorometers). In some aspects, the sensor 100 may include one or more chemical or biochemical sensors. In some aspects, the sensor 100 may be a radio frequency identification (RFID) device. In some aspects, the sensor 100 may be a small, fully subcutaneously implantable sensor that detects the presence, amount, and/or concentration of an analyte (e.g., glucose, oxygen, cardiac markers, low-density lipoprotein (LDL), high-density lipoprotein (HDL), or triglycerides) in a medium (e.g., interstitial fluid) of a living animal (e.g., a living human). However, this is not required, and, in some alternative aspects, the sensor 100 may be a partially implantable (e.g., transcutaneous) device or a fully external sensor. In addition, although aspects of the invention are described with respect to an analyte monitoring system in which the sensor 100 is an analyte sensor, this is not required. In some alternative aspects, the sensor 100 is different type of device or apparatus, such as, for example and without limitation, an insulin pump (e.g., an implantable insulin pump), a pacemaker (e.g., an implantable pacemaker), or electrical/heat therapy device (e.g., an implantable electrical/heat therapy device).

In some aspects, the external device 101 may be an externally worn device (e.g., attached via an armband, wristband, waistband, or adhesive patch). In some aspects, the external device 101 may remotely communicate with the sensor 100 (e.g., via near field communication (NFC)). In some aspects, the external device 101 may communicate with the sensor 100 to initiate and/or read data (e.g., measurements) from the sensor 100. In some aspects, the external device 101 may be a transceiver. In some aspects, the external device 101 may be a smartphone (e.g., an NFC-enabled smartphone). In some aspects, the external device 101 may communicate information (e.g., one or more analyte measurements) wirelessly (e.g., via a Bluetooth™ communication standard such as, for example and without limitation Bluetooth Low Energy) to an application running on a display device 107 (e.g., smartphone). In some aspects, the display device 107 may additionally or alternatively communicate directly with the sensor 100 (e.g., via near field communication (NFC)). In some aspects, the display device 107 may communicate with the sensor 100 to initiate and/or read data (e.g., measurements) from the sensor 100.

In some aspects, the external device 101 may be an electronic device that communicates with the sensor 100 to power the sensor 100, provide commands and/or data to the sensor 100, and/or receive data from the sensor 100. For example, in some aspects, the external device 101 may convey data by modulating the electromagnetic wave generated by the inductive element 105 (e.g., by modulating the current flowing through the inductive element 103 of the external device 101). In some aspects, the received data may include one or more sensor measurements. In some aspects, the sensor measurements may include, for example and without limitation, one or more light measurements from one or more photodetectors of the sensor 100 and/or one or more temperature measurements from one or more temperature sensors of the sensor 100. In some aspects, the external device 101 may receive data by detecting modulations in the electromagnetic wave generated by the sensor 100, e.g., by detecting modulations in the current flowing through the inductive element 103 of the external device 101. In some aspects, the external device 101 may calculate analyte (e.g., glucose) concentrations from the measurement information received from the sensor 100.

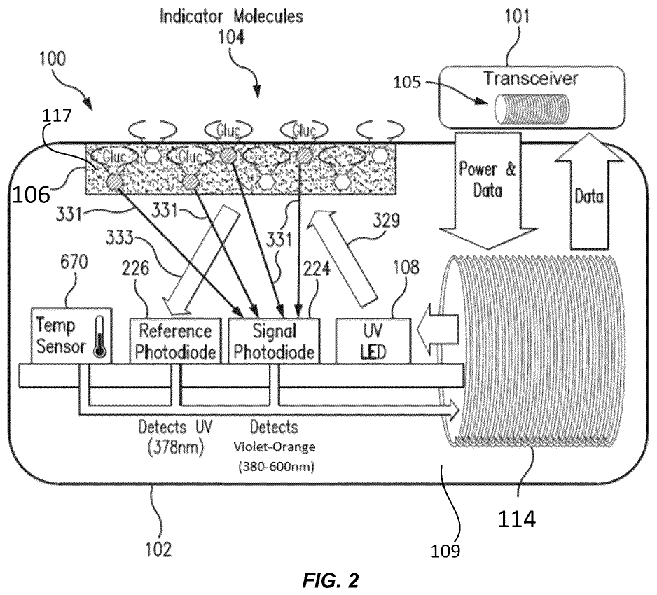

FIG. 2 is a schematic view of a system embodying aspects of the present invention. In some aspects, as shown in FIG. 2, the external device 101 may include an inductive element 105, such as, for example, a coil. In some aspects, the external device 101 may generate an electromagnetic wave or electrodynamic field (e.g., by using a coil) to induce a current in an inductive element 115 of the sensor 100. In some aspects, the sensor 100 may use the current induced in the inductive element 115 to power the sensor 100. However, this is not required, and, in some alternative aspects, the sensor 100 may be powered by an internal power source (e.g., a battery).

In some aspects, as shown in FIG. 2, the sensor 100 may include a sensor housing 102 (e.g., body, shell, capsule, or encasement), which may be rigid and biocompatible. In aspects, sensor housing 102 may be formed from a suitable, optically transmissive polymer material, such as, for example, acrylic polymers (e.g., polymethylmethacrylate (PMMA)).

In some aspects, the sensor 100 may include analyte indicator material 117 in one or more sensing areas (e.g., a first sensing area 106). In some aspects, the analyte indicator material 117 may be, for example, a polymer graft or hydrogel coated, diffused, adhered, embedded, or grown on or in at least the portion of the exterior surface of the housing 102 in at least the first sensing area 106. In some aspects, the analyte indicator material 117 may include analyte indicator molecules 104, which may be distributed throughout the analyte indicator material 117. In some aspects, the analyte indicator molecules 104 in the first sensing area 106 may have one or more detectable properties (e.g., optical properties) that vary in accordance with the amount or concentration of an analyte in proximity to the first sensing area 106. In some aspects, the analyte indicator molecules 104 may be, for example, fluorescent analyte indicator molecules. In some aspects, the analyte indicator molecules 104 may be phenylboronic-based analyte indicators. However, a phenylboronic-based analyte indicator is not required, and, in some alternative aspects, the analyte indicator material 117 may include different analyte indicator molecules 104, such as, for example and without limitation, glucose oxidase-based indicators, glucose dehydrogenase-based indicators, and glucose binding protein-based indicators. In some aspects, the analyte indicator molecules 104 in the analyte indicator material 117 may be selected from a group including fluorescent indicator molecules (e.g., TFM. having the chemical name 9-[N-[6-(4,4,5,5,-tetramethyl-l,3,2-dioxaborolano)-3-(trifluoromethyl)benzyl]-N-[3- (methacrylamido)propylamino]methyl]-10-[N-[6-(4,4,5,5,-tetramethyl-l,3,2-dioxaborolano)-3-(trifluoromethyl)benzyl]-N-[2-(carboxyethyl)amino]methyl]anthracene sodium salt) or light absorbing, non-fluorescent indicator molecules.

In some aspects, the sensor 100 may include one or more light sources 108, which may be, for example, one or more light emitting diodes (LEDs) or other light sources that emit radiation, including radiation over a range of wavelengths that interact with the analyte indicator molecules 104. In some aspects, the light source 108 may emit the excitation light 329 that irradiates the analyte indicator molecules 104 in the analyte indicator material 117. In some aspects, the light source 108 may emit excitation light 329, for example, at a wavelength of approximately 378 nm.

In some aspects, the sensor 100 may also include one or more photodetectors (e.g., photodiodes, phototransistors, photoresistors or other photosensitive elements). For example, as illustrated in FIG. 2, the sensor 100 may include one or more first photodetectors 224 and one or more second photodetectors 226. However, this is not required, and, in some alternative aspects, the sensor 100 may only include the one or more first photodetectors 224. In the case of a fluorescence-based sensor, the one or more first photodetectors 224 may be sensitive to fluorescent light emitted by the indicator molecules 104 such that a signal is generated by a first photodetector 224 in response thereto is indicative of the level of fluorescence of the analyte indicator molecules 104 and, thus, the amount of analyte of interest (e.g., glucose).

In some aspects, a part of the excitation light 329 emitted by the light source 108 may be reflected from the analyte indicator material 117 of the first sensing area 106 back into the sensor 100 as reflection light 333, and the analyte indicator molecules 104 may emit emission light 331 in response to being irradiated with a part of the excitation light 329. In some aspects, the emitted light 331 may have a different wavelength than the wavelength of the excitation light 329. In some aspects, the reflected light 333 and the emitted (e.g., fluoresced) light 331 may be absorbed by the first and second photodetectors 224 and 226, respectively, within the housing 102 of the sensor 100.

In some aspects, each of the one or more photodetectors may be covered by a filter that allows only a certain subset of wavelengths of light to pass through. In some aspects, the one or more filters may be thin glass filters. In some aspects, the one or more filters may be thin film (e.g., dichroic) filters deposited on the glass and may pass only a narrow band of wavelengths and otherwise reflect most of the received light. In some aspects, the filters may be thin film (dichroic) filters deposited directly onto the photo detectors and may pass only a narrow band of wavelengths and otherwise reflect most of the light received thereby.

In some aspects, the filter over the one or more second photodetectors 226 may allow the reflected excitation light 333 to pass through, and the filter over the one or more first photodetectors 224 may allow the emission light 331 to pass through. In some aspects, the one or more second photodetectors 226 may detect an amount of excitation light 333 that is reflected from the analyte indicator material 117. In some aspects, the one or more first photodetectors 224 may detect an amount of emission light 331 that is emitted from the analyte indicator molecules 104 in the analyte indicator material 117 of the first sensing area 106. In some aspects, the peak emission of the indicator molecules 104 may occur around 435 nm, and the one or more first photodetector 224 may be covered by a signal filter that passes light in the range of about 380 nm to 600 nm. In some aspects, higher glucose levels/concentrations correspond to a greater amount of emission light 331 from the indicator molecules 104 in the analyte indicator material 117, and, therefore, a greater number of photons striking the one or more first photodetectors 224.

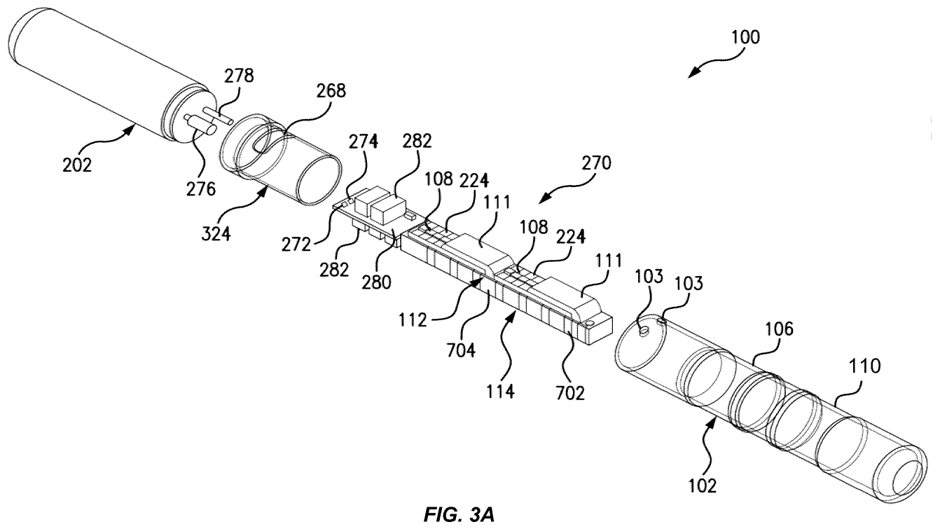

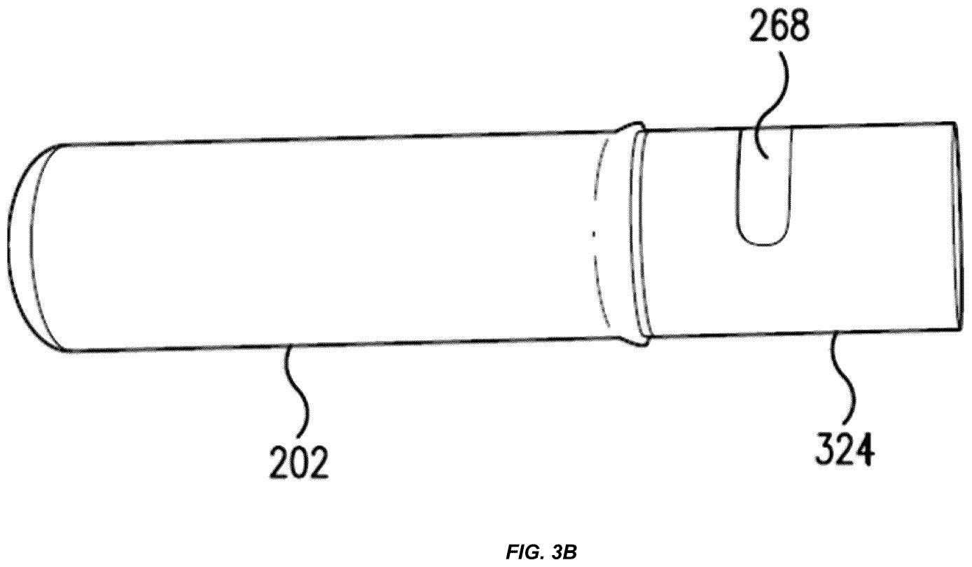

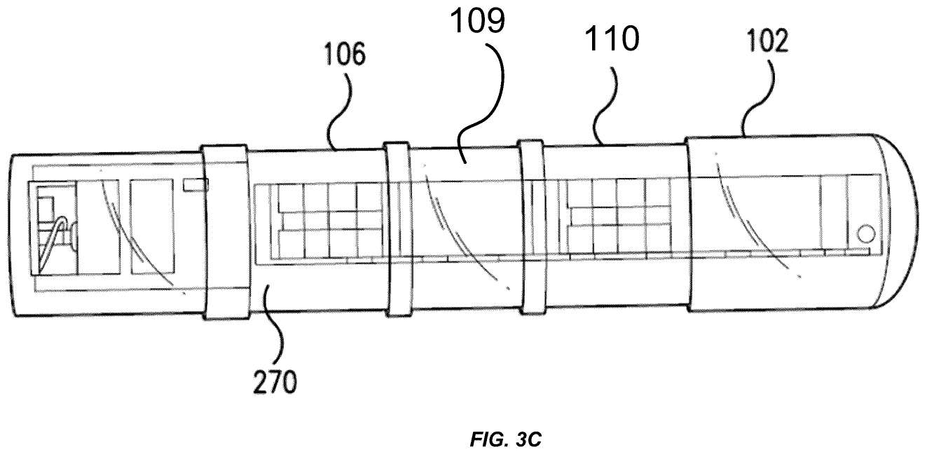

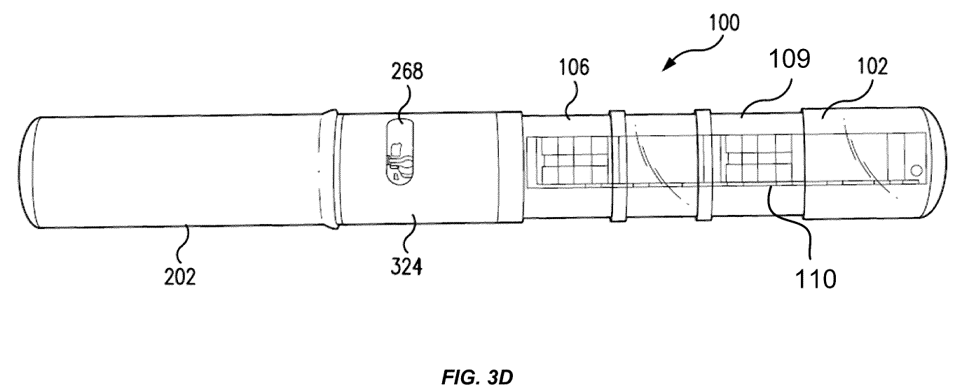

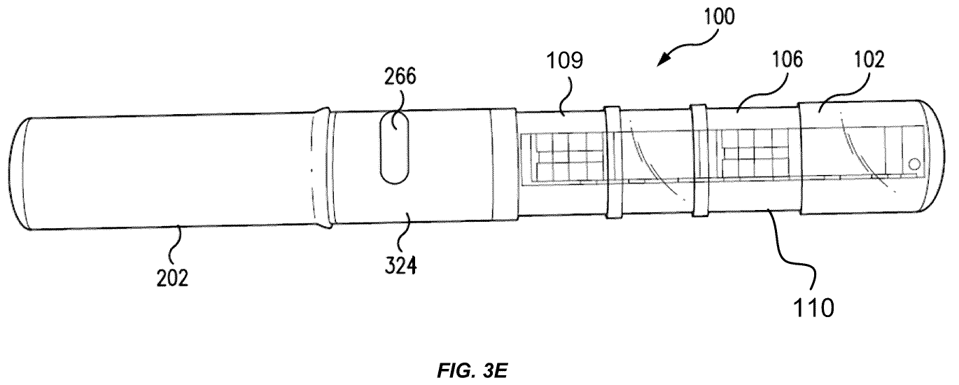

FIG. 3A is an exploded view of the sensor 100 of the system 50 according to some aspects. In some aspects, as shown in FIG. 3A, the sensor 100 may include the housing 102, circuitry 270, a power source 202, first and second electrically conductive leads 276 and 278, and/or a coupler 324. In some aspects, as shown in FIG. 3B, a first end of the coupler 324 may be attached to the power source 202. In some aspects, as shown in FIG. 3C, the circuitry 270 may be at least partially within the housing 102. In some aspects, as shown in FIGS. 3D and 3E, at least a portion of the housing 102 may extend into a second end of the coupler 324.

In some aspects, as shown in FIG. 2, the sensor 100 may include a single sensing area (e.g., first sensing area 106). However, this is not required, and, in some alternative aspects, the sensor 100 may include multiple sensing areas (e.g., two sensing areas 106 and 110 as shown in FIGS. 3A and 3C-3E). In some aspects, the sensor 100 may include, in addition to analyte indicator material 117 including analyte indicator molecules 104 in or on the housing 102 in the first sensing area 106, analyte indicator material 117 including analyte indicator molecules 104 in or on the housing 102 in a second sensing area 110. In some aspects, the analyte indicator material 117 in in the second sensing area 110 may be, for example, a polymer graft or hydrogel coated, diffused, adhered, embedded, or grown on or in at least the portion of the exterior surface of the housing 102 in the second sensing area 110.

In some aspects, as shown in FIGS. 3A and 3C-3E, the housing 102 may include one or more cutouts or recesses in the first and/or second sensing areas 106 and 110. In some aspects, the analyte indicator material 117 may be located (partially or entirely) in the cutouts or recesses. In some aspects, the analyte indicator material 117 in the first and second sensing areas 106 and 110 may be porous and may allow an analyte (e.g., glucose) in a medium (e.g., interstitial fluid) to diffuse into the analyte indicator material 117. In some aspects, the analyte indicator material 117 in the first sensing area 106 may be the same as the analyte indicator material 117 in the second sensing area 110. However, this is not required, and, in some alternative aspects, the analyte indicator material 117 in the first sensing area 106 may be different than the analyte indicator material 117 in the second sensing area 110.

In some aspects, the circuitry 270 may include measurement electronics (e.g., optical measurement electronics), one or more circuit components 111 (e.g., analog and/or digital circuit components), an antenna 114, one or more capacitors 282, and/or first and second contact pads 272 and 274. In some aspects, the measurement electronics of the circuitry 270 may include one or more light sources 108 (e.g., one or more light emitting diodes (LEDs)) and one or more photodetectors 224 (e.g., one or more photodiodes, phototransistors, photoresistors, or other photosensitive elements). In some aspects, the one or more light sources 108 may be configured to emit excitation light 329 (e.g., ultraviolet (UV) light) that reaches the analyte indicator material 117 of a sensing area (e.g., first sensing area 106 or second sensing area 110). In some aspects, the one or more photodetectors 224 may be configured to detect emission light 331 (e.g., fluorescent light) that reaches the one or more photodetectors 224 after being emitted by the indicator molecules 104 of the analyte indicator material 117. In some aspects, the amount of emission light emitted by the indicator molecules 104 of the analyte indicator material 117 may correspond to the amount of analyte (e.g., glucose) in the medium (e.g., interstitial fluid) in proximity to the first sensing area 106 and/or the second sensing area 110. For example, in some aspects, the analyte may bind reversibly to analyte indicator molecules 104, the analyte indicator molecules 104 may emit emission light 331 when irradiated by the excitation light 329, and analyte indicator molecules 104 to which the analyte is not bound may not emit light (or emit only a small amount of light) when irradiated by the excitation light 329.

In some aspects, as shown in FIG. 3A, the sensor 100 may include one or more substrates 112. In some aspects, the one or more substrates 112 may be circuit boards (e.g., one or more flexible and/or rigid printed circuit boards (PCBs)). In some aspects, one or more of the circuit components 111 may be mounted or otherwise attached to the one or more substrates 112. However, in some alternative aspects, the one or more substrates 112 may be semiconductor substrates having one or more of the circuit components 111 fabricated therein. For instance, the fabricated circuit components may include analog and/or digital circuitry. Also, in some aspects in which the substrate 112 is a semiconductor substrate, in addition to the one or more circuit components fabricated in the semiconductor substrate, one or more circuit components may be mounted or otherwise attached to the semiconductor substrate. In other words, in some semiconductor substrate aspects, a portion or all of the circuit components 111, which may include discrete circuit elements, an integrated circuit (e.g., an application specific integrated circuit (ASIC)) and/or other electronic components (e.g., a non-volatile memory), may be fabricated in the semiconductor substrate with the remainder of the circuit components 111 secured to the semiconductor substrate, which may provide communication paths between the various secured components.

In some aspects, as shown in FIG. 3A, the measurement electronics of the circuitry 270 may be mounted on and/or fabricated in the one or more substrates 112. In some aspects, as shown in FIG. 3A, the one or more substrates 112 may include (i) a first set of one or more light sources 108 and one or more photodetectors 224 and (ii) a second set of one or more light sources 108 and one or more photodetectors 224. In some aspects, the one or more light sources 108 may be mounted on the one or more substrates 112, the one or more photodetectors 224 may be fabricated in the substrate 112, and all or a portion of the circuit components 111 may be fabricated within the substrate 112.

In some aspects, as shown in FIG. 3A, the antenna 114 may be an inductor including a conductor 702 in the form of a coil and a magnetic core 704. In some aspects, the core 704 may be, for example and without limitation, a ferrite core. In some aspects, the antenna 114 may be, for example, a ferrite-based micro-antenna. In some aspects, as illustrated in FIG. 3A, the one or more substrates 112 of the sensor 100 may be attached to the antenna 114. In some aspects, the circuit components 111 of the substrates 112 may be connected electrically to the antenna 114. In some aspects, the sensor 100 may use the antenna 114 to communicate data (e.g., measurement data) to the external device 101 and/or the display device 107. In some aspects, the sensor 100 may use the antenna 114 for NFC.

In some aspects, as shown in FIG. 3A, the sensor 100 may include a PCB 280. In some aspects, the one or more capacitors 282 of the circuitry 270 may be mounted on the PCB 280. In some aspects, the PCB 280 may include the first and second contact pads 272 and 274 of the circuitry 270. In some aspects, the circuit components 111 of the substrates 112 and/or the antenna 114 may be connected electrically to the one or more capacitors 282 and/or the first and second contact pads 272 and 274.

In some aspects, the sensor 100 (e.g., the circuitry 270 of the sensor 100) may be powered at least partially by the power source 202. In some aspects, the power source 202 may be a charge storage device (e.g., a battery, capacitor, or super capacitor). In some aspects, at least the exterior of the power source 202 may be made of a biocompatible material such as, for example and without limitation, stainless steel or a titanium alloy. In some aspects, the power source 202 may be a titanium-cased, hermetically-sealed battery. In some aspects, as shown in FIGS. 3A, 3D, and 3E, the circuitry 270 of the sensor 100 may extend away from the power source 202 along the longitudinal axis of the power source 202.

In some aspects, the power source 202 may include first and second terminals (e.g., a positive terminal (cathode) and a negative terminal (anode)). In some aspects, the first and second electrically conductive leads 276 and 278 may be connected electrically to the first and second terminals, respectively, of the power source 202. In some aspects, the electrically conductive leads 276 and 278 may electrically connect the first and second terminals, respectively, of the power source 202 to the circuitry 270 of the sensor 100. In some aspects, the electrically conductive leads 276 and 278 may be rods or beams including or made out of a conductive material.

In some aspects the analytical sensor does not have an internal power source, but is powered through induction by an external power source.

In some aspects, as shown in FIGS. 3A, 3B, 3D, and 3E, the coupler 324 may be a flange. In some aspects, as shown in FIGS. 3B, 3D, and 3E, the coupler 324 may be attached to the power source 202. In some aspects, the coupler 324 may be welded (e.g., laser welded) to the power source 202. In some aspects, the coupler 324 may enclose the first and second terminals of the power source 202. In some aspects, as shown in FIGS. 3D and 3E, the coupler 324 may be between the housing 102 and the power source 202. In some aspects, as shown in FIG. 3E, the sensor 100 may further include a cap 266 over the one or more openings 268 of the coupler 324.

In some aspects, the coupler 324 may have a generally cylindrical shape. However, other shapes (e.g., a generally rectangular prism shape) may be used in alternative aspects. In some aspects, the coupler 324 may be made of a biocompatible material such as, for example and without limitation, glass, ceramic, stainless steel, titanium, or a titanium alloy. In some aspects, the coupler 324 may include a flat surface that abuts and is attached to the power source 202.

In some aspects, as shown in FIGS. 3A, 3B, 3D, and 3E, the coupler 324 may include one or more openings 268 through which the first and second electrically conductive leads 276 and 278 are capable of being laser welded to the first and second contact pads 272 and 274, respectively, of the circuitry 270. In some aspects, the housing 102 may include one or more openings 103 through which the first and second electrically conductive leads 276 and 278 are capable of being laser welded to the first and second contact pads 272 and 274, respectively, of the circuitry 270.

In some aspects, as shown in FIG. 3E, the sensor 100 may further include an encasement material 109 that encases at least a first portion of the circuity 270 in the housing 102. In some aspects, the first portion of the circuitry 270 may include the one or more light sources 108 and the one or more photodetectors 224. In some aspects, the encasement material 109 may include a water-resistant epoxy.

In some aspects, the encasement material 109 may be a first encasement material that encases the first portion of the circuitry 270, and the first portion of the circuitry may not include the first and second contact pads 272 and 274. In some aspects, the sensor 100 may further include a second encasement material that encases the first and second electrically conductive leads 276 and 278 and a second portion of the circuitry 270. In some aspects, the second portion of the circuitry may include the first and second contact pads 272 and 274. In some aspects, the first and second encasement materials may be different. In some alternative aspects, the first and second encasement materials may be the same. In some aspects, the second encasement material may include a water-resistant epoxy. In some aspects, the first encasement material may fill a first portion of the housing 102, and the second encasement material may fill the coupler 324 and a second portion of the housing 102 that is not filled by the first encasement material.

In some alternative aspects, instead of first and second encasement materials, the encasement material may include a single encasement material that encases the circuitry 270 and the first and second electrically conductive leads 276 and 278. In some aspects, the encasement material may fill the housing 102 and the coupler 324.

In some aspects, the excitation light emitted by the one or more light sources 108 of the circuitry 270 may reach the first sensing area 106 and/or the second sensing area 110 after passing through the encasement material (e.g., the first encasement material or the single encasement material). In some aspects, the emission light emitted by the first sensing area 106 may reach the one or more photodetectors 224 after passing through the encasement material (e.g., the first encasement material or the single encasement material).

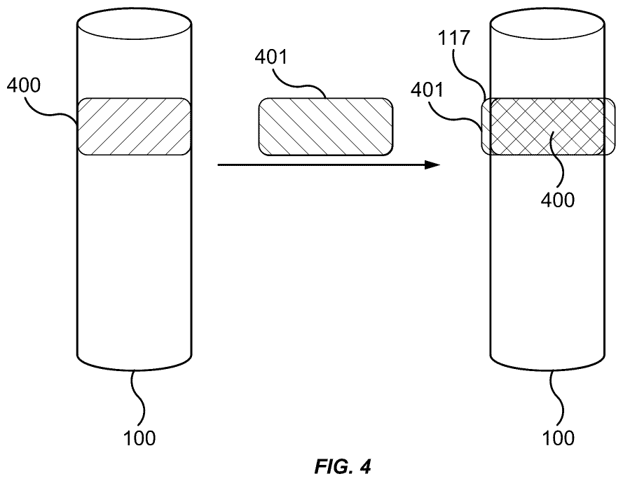

In some aspects, as shown in FIGS. 4-7C, the analyte indicator material 117 of the sensor 100 (e.g., in the first sensing area 106 and/or the second sensing area 110) may include a first polymer 400 and a second polymer 401. In some aspects, the first polymer 400 cover at least a portion of the housing 102. In some aspects, the first polymer 400 may be a hydrogel. In some aspects, second polymer 401 may be a hydrogel. In some aspects, the first polymer 400 may include the indicator molecules 104.

In some aspects, the second polymer 401 may form an interpenetrating network (IPN) with the first polymer 400. An interpenetrating network is a structure in which one or more polymers exist within the same matrix but are entangled on a molecular level. An interpenetrating network may, in some aspects, prevent protein or other biological materials from sticking to or diffusing into the sensor. This property may protect the system from biological reactions or from optical interference derived from biological materials. In some aspects, the polymers 400 and 401 of the interpenetrating network may not be covalently bonded. In such an aspect, the network allows the first polymer 400 and the second polymer 401 to maintain distinct chemical identities while physically intertwining. In some aspects, the second polymer 401 may be physically interweaved or interwoven with the first polymer 400. In some aspects, the second polymer may be interpenetrated with the first polymer 400. In some aspects, the second polymer 401 may be linked to the first polymer 400. In some aspects, the second polymer 401 may be chemically linked (e.g., covalently linked) to the first polymer 400. In some aspects, the second polymer 401 may be physically linked to the first polymer 400. In some aspects in which the second polymer 401 is physically linked to the first polymer 400, the physical link between the first and second polymer may be through physical entanglement. In some aspects, the second polymer 401 may be linked to the first polymer 400. In some aspects, the second polymer 401 may be linked to the surface of the first polymer 400. In some alternative aspects, the second polymer 401 may be linked to the first polymer 400 other than by the surface of the first polymer 400. In some aspects, the second polymer 401 may also include analyte indicator molecules 104, but this is not required, and, in some alternative aspects, the second polymer 401 may not include analyte indicator molecules. In some aspects, the second polymer 401 may be a non-modulatory hydrogel. In some aspects, the second polymer 401 may reduce absorption and/or adsorption of one or more proteins to the first polymer 400. In some aspects, the second polymer 401 may reduce absorption and/or adsorption of one or more macrophages to the first polymer 400. In some aspects, the second polymer 401 may reduce absorption and/or adsorption of one or more bacteria to the first polymer 400. In some aspects, the second polymer 401 may reduce biological reactions and/or optical interference of the sensor 100. In some aspects, the second polymer 401 may reduce chemical degradation and/or oxidation of the analyte indicator molecules 104 of the analyte indicator material 117 (e.g., relative to an analyte indicator material 117 that includes the first polymer 400 and does not include the second polymer 401).

In some aspects, the second polymer 401 may be grown from the first polymer 400. In some alternative aspects, the second polymer 401 may be formed independently of the first polymer 400. In some alternative aspects, the first polymer 400 and the second polymer 401 may be formed simultaneously. In some alternative aspects, the first polymer 400 and the second polymer 401 may not be formed simultaneously. In some aspects, the second polymer 401 may include single linear polymer chains. In some alternative aspects, the second polymer 401 may include cross-linked portions of polymer chains. In some alternative aspects, the second polymer 401 may be formed interweaved or interwoven with the first polymer 400 through a portion of its geometry. In some alternative aspects, the second polymer 401 may be formed interweaved or interwoven with the first polymer 400 through its entire geometry.

In some aspects, at least the first polymer 400 of the analyte indicator material 117 may be applied (e.g., coated, deposited, diffused, adhered, or embedded) on at least a portion (e.g., one or more sensing areas 106 and 110) of the exterior surface of the sensor housing 102. In some aspects, the first polymer 400 may cover the entire surface of sensor housing 102 or only one or more portions (e.g., one or more sensing areas 106 and 110) of the exterior surface of housing 102. In some aspects, as an alternative to coating the first polymer 400 on the outer surface of sensor housing 102, the first polymer 400 may be disposed on the outer surface of the sensor housing 102 in other ways, such as by deposition or adhesion. In some aspects, the analyte indicator material 117 may be a fluorescent glucose indicator material. In some aspects, the analyte indicator material 117 may be biocompatible and stable, grafted onto the surface of sensor housing 102, and configured to allow for the measurement of glucose in interstitial fluid (ISF), blood, or intraperitoneal fluid after implantation of the sensor 100.

In some aspects, the first polymer 400 may include co-monomers of four monomers according to Formula Ia: ABCD [Formula Ia]. In some aspects, A may be an analyte indicator monomer. In some aspects, B may be a methacrylate monomer. In some aspects, C may be a polyethylene glycol (PEG) monomer. In some aspects, D may be a compound or monomer including boronate or boronic acid containing moieties. In some aspects, A may be 0.001%, 0.005%, 0.01%, 0.05%, 0.1 %, 0.2 %, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 20%, 25%, 30% or greater by weight of Formula Ia. In some aspects, A may be between 0.001% to 30% by weight of Formula Ia. In some aspects, A may be between 0.005% to 20% weight of Formula Ia. In some aspects, A may be between 0.01% to 10% by weight of Formula Ia. In some aspects, B may be between 0.01% to 99.9% by weight of Formula Ia. In some aspects, B may be between 0.1% to 99.5% by weight of Formula Ia. In some aspects, B may be between 1% to 99 % by weight of Formula Ia. In some aspects, C may be between 0.01% to 99.9% by weight of Formula Ia. In some aspects, C may be between 0.1% to 99.5% by weight of Formula Ia. In some aspects, C may be between 1 to 99 % by weight of Formula Ia. In some aspects, D may be between 0.01 to 99% by weight of Formula Ia.

In some aspects, the PEG of Formula Ia may be polyethylene glycol methacrylate (PEG-methacrylate) or polyethylene glycol diacrylate (PEG-diacrylate or PEGDA). In some aspects, the boronate or boronic acid containing moieties monomer of Formula Ia may be methacrylate-containing phenyl boronic acid or boronate-containing moieties. In some aspects, the monomers may be in specific molar ratios. For example, in some aspects in which the first polymer 106 may be opaque, HEMA may be 10 to 90 molar percent, PEGDA may be 10 to 90 molar percent, and the methacrylate-containing phenyl boronic acid or boronate-containing moieties may be 0.001 to 90 molar percent.

In some aspects, the PEGDA may act as a cross-linker and create a sponge-like matrix/hydrogel. In some aspects, the PEG-containing graft/hydrogel may become clear if a sufficient amount of additional PEG is added to the mixture (e.g., if it is fabricated with a higher concentration of PEG), and a clear hydrogel may be made from such a formulation. For example, in some aspects, the hydrogel may be made using a polymer solution that is 50-60% water by volume and 40-50% monomers by volume, where the HEMA, PEG-methacrylate, and the compound containing boronate or boronic acid containing moieties may comprise 0.01 to 10 %, 1 to 99 %, 1 to 99 %, and 0.01 to 99% by weight, of the monomers in the solution. In some aspects, the polymer graft may be synthesized using conventional free radical polymerization.

In some aspects, the second polymer 401 may include one or more co-monomers. In some aspects, the second polymer 401 may include co-monomers of three monomers according to Formula Ib: EFG [Formula Ib].

In some aspects, the co-monomers of the second polymer may be covalently linked via free radical polymerization, click chemistry or step growth polymerization. In some aspects, the co-monomers of the second polymer may be joined by ionic bridging, or physical network formation. In some aspects, the co-monomers include 2-Methacryloyloxyethyl phosphorylcholine (MPC), poly(carboxybetaine methacrylate) (poly(CBMA)), polysulfobetaine methacrylate, or sulfobetaine methacrylate. In some aspects, the co-monomers may be covalently linked via click chemistry. In some aspects, the click chemistry may be used with or initiators, catalysts, light, or temperature to catalyze the reaction. In some aspects, the click chemistry may be used without or initiators, catalysts, light, or temperature to catalyze the reaction. In some aspects, the co-monomers covalently linked via click chemistry may comprise thiols, maleimides, norbornenes, vinylsulfones, orthopyridyl sulfides, acrylate, acrylamide, carbonylacrylic, amines, hydroxyls, n-hydroxysuccinimide (NHS) ester derivatives, and/or epoxides.

In some aspects, E may be a methacrylate monomer. In some aspects, F may be a polyethylene glycol monomer. In some aspects, G may be a compound or monomer including boronate or boronic acid containing moieties.

In some aspects, the F monomers of the second polymer may be thiolene polymerized. In some aspects, the F monomers of the second polymer may comprise N-hydroxysuccinimide (NHS), dibenzylcyclooctyne (DBCO), amine, epoxide, vinylsulfone, malemide, norebornene, thiol, azide, or alkyne groups. In some aspects, E may be between 0.01% to 99.9% by weight of Formula Ib. In some aspects, E may be between 0.1% to 99.5% by weight of Formula Ib. In some aspects, E may be between 1% to 99 % by weight of Formula Ib. In some aspects, F may be between 0.01% to 99.9% by weight of Formula Ib. In some aspects, F may be between 0.1% to 99.5% by weight of Formula Ib. In some preferred aspects, F may be between 1% to 99 % by weight of Formula Ib. In some aspects, G may be between 0.01% to 99% by weight of Formula Ib.

In some aspects, the analyte indicator molecules 104 of the first polymer 400 may emit emission light 331 in response to being irradiated by excitation light 329, and an amount of the emission light 331 may vary in accordance with an amount or concentration of analyte in proximity to the first polymer 400. In some aspects, the second polymer 401 may affect neither the amount of the emission light nor an ability of the analyte to reach the first polymer 400.

In some aspects, the chemical degradation and/or oxidation of the analyte indicator molecules 104 of the analyte indicator material 117 may be detrimental to the functioning of the senor 100 and may result from adverse physiological reactions that may be exhibited by a user/patient's body following implantation or insertion of the sensor 100 into the user/patient's body. The reactions may range from infections due to implantation surgery to the immunological response of a foreign object implanted in the body. That is, the performance of the sensor 100 may be hindered or permanently damaged in vivo via the immunological response to an infection or the sensor 100 itself. In particular, the performance of the indicator molecules 104 of the analyte indicator material 117 may be deteriorated by the immunological response of the body into which the sensor 100 is implanted. For example, white blood cells, including neutrophils, may attack an implanted sensor 100. The neutrophils release, inter alia, hydrogen peroxide, which may degrade indicator molecules 104 (e.g., by oxidizing a boronate group of an indicator molecule 104 and disabling the ability of the indicator molecule 104 to bind glucose). Further, proteins, macrophages, and other types of cells and cellular materials may attach to, react with, or be absorbed by the analyte indicator material 117 leading to immunogenicity, biofouling, and reduced biocompatibility. Further, in some aspects, infection and bacterial colonization on the analyte indicator material 117 may require the implanted sensor 100 to be removed. As noted above, in some aspects, the second polymer 401 may reduce chemical degradation and/or oxidation of the analyte indicator molecules 104 of the analyte indicator material 117.

In some aspects, the sensor 100 may include a plurality of reactive oxygen species (ROS) scavenger molecules covalently linked to the first polymer 400. In some aspects, the sensor 100 may include a plurality of ROS scavenger molecules covalently linked to the second polymer 401. In some aspects, the ROS scavenger molecules may be covalently linked via monomer spacers, which may be flexible and hydrophilic. In some aspects, the second polymer 401 may reduce absorption or adsorption of one or more proteins to the first polymer 400. In some aspects, the second polymer 401 may reduce absorption or adsorption of one or more macrophages to the first polymer 400. In some aspects, the second polymer 401 may reduce absorption or adsorption of one or more bacteria to the first polymer 400. In some aspects, the second polymer 401 may reduce contact of degradative species with the first polymer 400. In some aspects, the degradative species may include hydrogen peroxide, a reactive oxygen species, a reactive nitrogen species, enzymes, free radicals, or metal ions. In some aspects, the second polymer 401 may not leach out of or dissociate from the sensor 100. In some aspects, the second polymer 401 may reduce, inhibit, or prevent electrostatic interactions of the first polymer 400 with one or more proteins.

FIG. 4 shows the sensor 100 of the system 50 according to some aspects. In some aspects, as shown in FIG. 4, the analyte indicator material 107 (e.g., in the first sensing area 106 and/or the second sensing area 110) may include the first polymer 400 and the second polymer 401. In some aspects, as shown in FIG. 4, during fabrication of the sensor 100, the second polymer 401 may be grown from or otherwise linked to the first polymer 400.

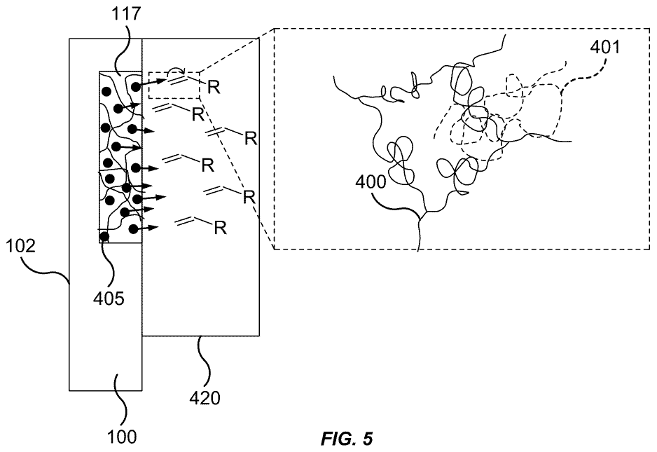

In some aspects, as shown in FIG. 5, growing the second polymer 401 from the first polymer 400 may include providing initiator molecules 405 to the first polymer 400. In some aspects, the initiator molecules 405 may be interspersed throughout the first polymer 400. In some aspects, the initiator molecules 405 may react as shown in FIG. 5 in a monomer solution 420 and result in the second polymer 401 being grown from the first polymer 400. In some aspects, as shown in FIG. 5, the initiator molecules 405 may diffuse outwards from the first polymer 400. In some aspects, the second polymer 401 grown from the first polymer 400 may form an interpenetrating network with the first polymer 400 as shown in FIG. 5.

In some aspects, the initiator molecules 405 may be selected from a group including free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amines, peroxide/ascorbic acid, ketone/amines), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

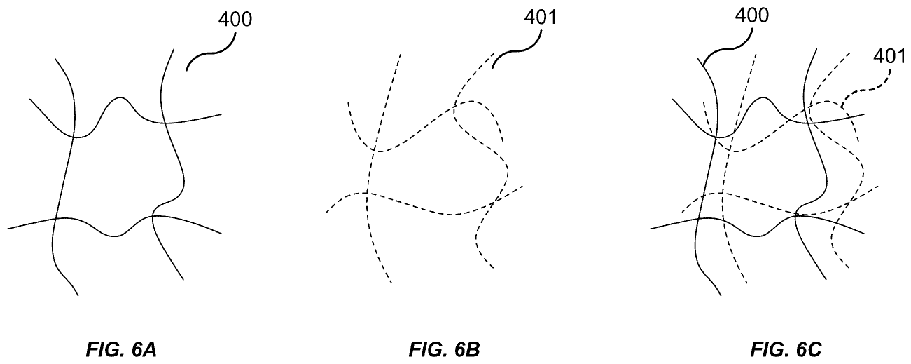

In some aspects, as shown in FIGS. 6A-6C, the first and second polymers 400 and 401 may be polymer networks. As shown in FIG. 6A, the first polymer 400 may be a polymer network, which may provide, according to some aspects, mechanical strength and the indicator molecules 104. In some aspects, the first polymer 400, depending on its composition and based on its proposed utility, may provide additional benefits including, but not limited to, improved thermal stability, improved chemical resistance, improved electrical conductivity, high modulus, and/or abrasion resistance.

As shown in FIG. 6B, the second polymer 401 may be a polymer network, which may provide, according to some aspects, biocompatibility and/or anti-oxidant properties. In some aspects, the second polymer 401, depending on its composition and based on its proposed utility, may provide additional benefits including, but not limited to, reduction of chemical degradation and/or oxidation, improved biodegradability, improved permeability, transparency, hydrophilicity, adhesion, and/or low toxicity.

FIG. 6C shows an interpenetrating network including the first polymer 400 and the second polymer 401. In some aspects, the interpenetrating network including the first polymer 400 and the second polymer 401. In some aspects, the interpenetrating network may provide the combined properties of each of the first and second polymers 400 and 401 individually (that is, in some aspects, strength, sensing elements, biocompatibility and anti-oxidant properties). In some aspects, the interpenetrating network may exhibit any combination of the properties of the first polymer 400 and the second polymer 401. In some aspects, the interpenetrating network including the first and second polymers 400 and 401 may offer a customizable platform where the selection and combination of first and second polymers 400 and 401 may be tailored to achieve the desired properties, either individually or synergistically, to meet a specific use case.

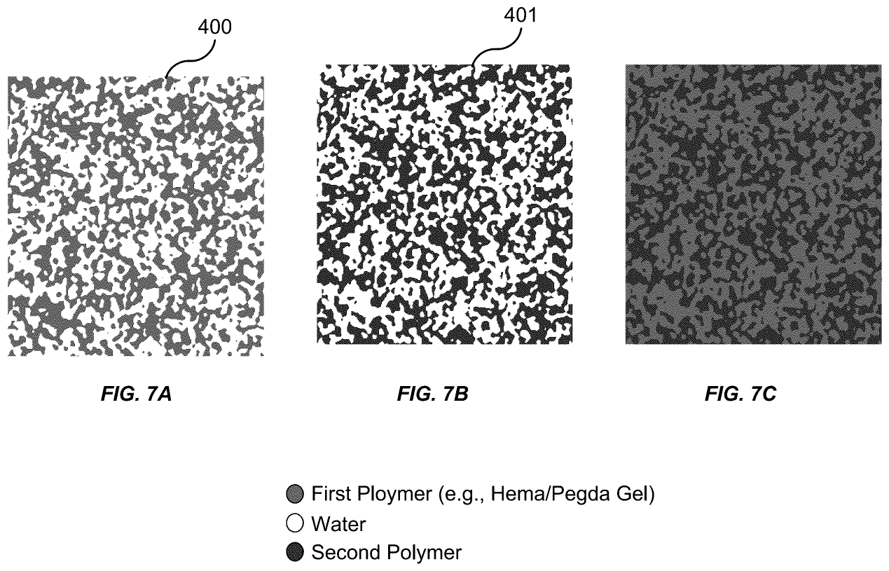

FIGS. 7A-7C show the first polymer 400, the second polymer 401, and an interpenetrating network including the first and second polymers 400 and 401, respectively, according to some aspects. In some aspects, the first polymer 400 may be, for example, a hydroxyethyl methacrylate (HEMA)/polyethylene glycol diacrylate (PEGDA) hydrogel microporous network. In some aspects, as shown in FIG. 7B, the second polymer 401 may be a polymer network on its own. In some aspects, as shown in FIG. 7C, in the interpenetrating network including the first polymer 400 and the second polymer 401, the network of the second polymer 401 may fill in pores of the network of the first polymer 401 to create the interpenetrating network of the analyte indicator material 117. In some alternative aspects, the interpenetrating network may include more than two polymer networks. For example, in some aspects, the interpenetrating network of the analyte indicator material 117 may include three, four, five, six or more, ten or more, or twenty or more polymer networks.

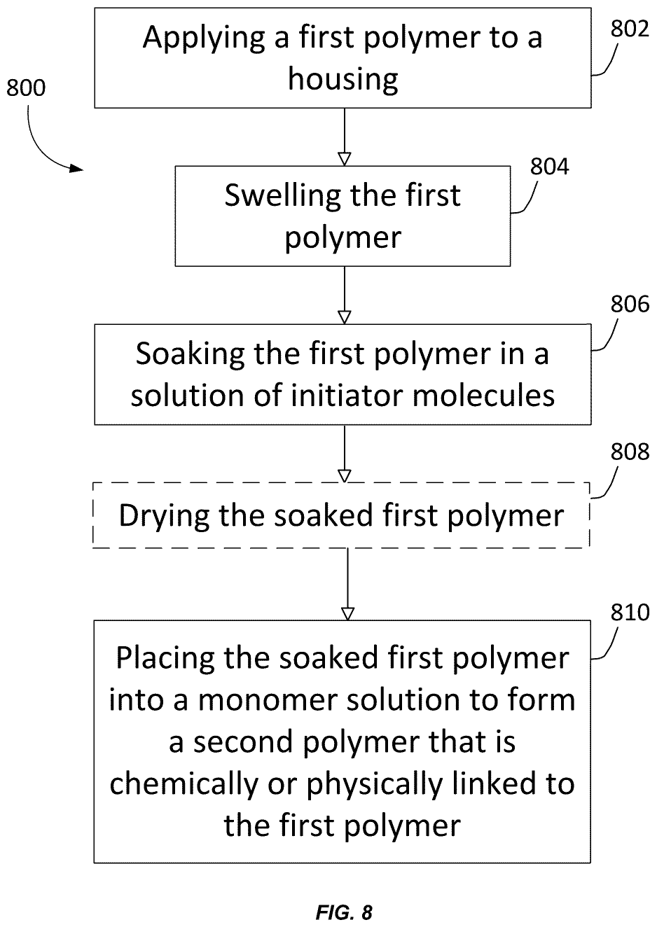

FIG. 8 is a flowchart illustrating a process 800 of fabricating a sensor 100 according to some aspects. In some aspects, as shown in FIG. 8, the process 800 may include a step 802 of applying a first polymer 400 to a housing 102 of the sensor 100. In some aspects, the first polymer 400 may be applied to the housing 102 such that the applied first polymer 400 covers at least a portion (e.g., a sensing area 106 or 110) of the housing 102. In some alternative aspects, the first polymer 400 may be applied to the housing 102 such that the applied first polymer 400 covers the entirety of the housing 102. In some aspects, the first polymer 400 may include analyte indicator molecules 104.

In some aspects, as shown in FIG. 8, the process 800 may include a step 804 of swelling the first polymer 400. In some aspects, swelling the first polymer 400 in step 804 may include allowing the polymer to absorb a solvent or other fluid to increase its volume. In some aspects, the amount or degree of swelling of the first polymer 400 in step 804 may be affected by, for example, the duration, the compatibility of the polymer and the solvent, the crosslink density of the polymer, the temperature of the solution and/or polymer, and/or the pH of the solution. In some aspects, swelling the first polymer 400 in step 804 may include swelling the first polymer 400 for a duration of time. In some aspects, the duration that the first polymer 400 is swelled in step 804 may control the extent to which the second polymer 401 is physically linked to the first polymer 400. In some alternative aspects, the duration that the first polymer 400 is swelled may be used to control the extent to which the second polymer 401 may be chemically linked to the first polymer 400.

In some aspects, as shown in FIG. 8, the process 800 may include a step 806 of soaking the first polymer 400 in a solution that includes indicator molecules. In some aspects, the step 806 may form a soaked first polymer. In some aspects, as shown in FIG. 8, the process 800 may include an optional step 808 of drying the soaked first polymer.

In some aspects, as shown in FIG. 8, the process 800 may include a step 810 of placing the first polymer 106 into a solution containing monomers (“monomer solution”) 420. In some aspects, the monomers may be monomers of a second polymer 401. In some aspects, the process 800 may form a second polymer 401 that forms an interpenetrating network with the first polymer 400. In some aspects, the process may form a second polymer 401 that is chemically or physically linked with the first polymer 400. In some aspects, as the first and second polymers 400 and 401 are formed in different steps, the first polymer 400 and the second polymer 401 may not be formed simultaneously. In some aspects, the solution may include a solvent. In some aspects, the solvent may control the extent to which the second polymer 401 is physically linked to the first polymer 400. In some alternative aspects, the solvent may control the extent to which the second polymer 401 may be chemically linked to the first polymer 400. In some aspects in which the process 800 includes the step 808, the step 808 of drying the soaked first polymer may be performed before the step 810 of placing the soaked first polymer 400 into the monomer solution 420 to form the second polymer 401. In some aspects, the monomer solution 420 may include a mixture of initiator molecules 405 and monomer molecules 104. In some alternative aspects, the process 800 may include multiple instances of the step 810 of placing the soaked first polymer 400 into a monomer solution 420 with different monomers (i.e., a first version of step 810, placing the soaked first polymer 400 into a first monomer solution and a second version of step 810, placing the soaked first polymer 400 into a second monomer solution) to form the second polymer 401. In some aspects of the process 800, the monomers solutions 420 are the same. In some aspects of the process 800, the monomer solutions 420 are not the same. In some aspects, the step may be performed without initiator molecules 405.

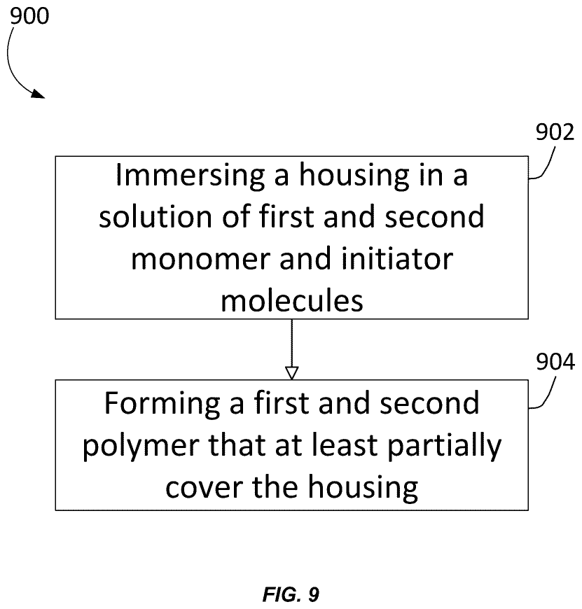

FIG. 9 is a flowchart illustrating a process 900 of fabricating the sensor 100 according to some aspects. In some aspects, as shown in FIG. 9, the process 900 may include a step 902 of immersing the housing 102 in a solution including first and second monomers, and initiator molecules 405.

In some aspects, the initiator molecules 405 may be selected from a group including free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amines, peroxide/ascorbic acid, ketone/amines), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

In some aspects, as shown in FIG. 9, the process 900 may include a step 904 of forming the first polymer 106 and the second polymer 110. In some aspects, the first polymer 400 and the second polymer 401 may be formed simultaneously. In some aspects, the first polymer 400 and the second polymer 401 may be formed at different rates. In some aspects, the first polymer may include analyte indicator molecules 104. In some aspects, the second polymer 401 may be chemically or physically linked to the first polymer 400. In some aspects, the first and second polymers 400, 401 may be formed such that the first and second polymers 400 and 401 at least partially cover the housing 102. In some aspects, the first and second polymers 400, 401 may be formed such that the first and second polymers 400, 401 entirely cover the housing 102.

Synthetic Procedure

In some aspects, an acrylate poly(ethylene glycol) amine (molecular weight 1000-5000 Daltons) may have been dissolved an aqueous solution at a concentration of 0.003-1 g per mL. In some aspects, other monomers may serve as mechanical reinforcement, such as acrylamide, (hydroxyethyl)methacrylate, acrylate poly(ethylene glycol), N, N'-Methylenebisacrylamide, and poly(ethylene diacrylate) can be dissolved between 0.25-1 g per mL. In some aspects, a thermal initiator, such as VAZO 44, may be dissolved in the monomer solution anywhere between 0.1-1 mg per mL. In some aspects, the monomer and initiation solution may be dissolved in aqueous buffer with acidic, neutral, or basic pH. In some aspects, polymerization may be performed between 40 and 65° C. for 0.5 to 5 hours.

In some aspects, to append a carboxylic acid catalytic molecule of interest, it may be dissolved in 0.1 2-(N-morpholino)ethanesulfonic acid buffer with a pH between 4 and 5. In some aspects, the carboxylic acid terminated catalytic molecule may be dissolved between 0.1 and 50 mM. In some aspects, the solution may be stirred for 15-30 minutes to ensure proper dissolution of the catalytic molecule. In some aspects, 1-Ethyl-3-[3-dimethylaminopropyl]carbodiimide hydrochloride (EDC) and N-hydroxysuccinimide (NHS) may be added excess to that of the carboxylic acid (e.g., EDC 0.5 mM and NHS 1 mM for 0.1 mM carboxylic acid solutions; EDC 100 and NHS 200 mM for 50 mM mM carboxylic acid solutions). In some aspects, the carboxylic acid and coupling reagents may mix for 15-30 minutes using a shaker or stir plate on medium-high settings. In some aspects, the solution pH may be brought up to neutral with 1N sodium hydroxide after the carboxylic acid/EDC/NHS reaction to ensure proper coupling to nucleophiles (primary amines, and hydroxyl functional groups). In some aspects, the hydrogel may be completely submerged in the carboxylic acid/EDC/NHS neutral pH solution for adequate coupling to the surface accessible amines or hydroxyls. In some aspects, once submerged, the coupling reaction may be placed on a shaker with medium-low settings for four hours. After the reaction is complete, the hydrogel may be rinsed of excess solution and may be placed in an aqueous buffer (e.g., phosphate buffer saline) to remove any unbound carboxylic acid or coupling reagents.

### Formation of Polymers

An exemplary procedure for forming some aspects of the disclosed sensor or sensor 100 is described below. It is understood that “grow” and “form” (as well as related tenses, like “grown” and “formed”) may be used to describe the processes by which a first polymer 400 and a second polymer 401 are made in a manner that results in their combination. Additional terms including “synthesize,” “polymerize,” “produce,” “develop,” and “generate” are further understood to describe the process of creating polymers are described herein.

In some aspects, the second polymer 401 may be grown from the first polymer 400. In such aspects, a first polymer 400 may be synthesized using a suitable polymerization method (for example, free radical polymerization or condensation polymerization). In such aspects, the synthesized first polymer 400 may be shaped or processed to form a suitable substrate or core. In such aspects, monomers of a second polymer 401, including but not limited to those described herein, may be introduced around the first polymer 400, and may be polymerized in situ. In some aspects, such a polymerization may occur via surface-initiated polymerization, initiator molecules 405 may be attached to the surface of the first polymer 400, and monomers of the second polymer 401 may be polymerized from said initiator molecule 405 attachment sites. An exemplary schematic shown in FIG. 4. In some alternative aspects, polymerization may occur via an encapsulation technique, whereby a first polymer 400 may be immersed in a solution containing monomers of the second polymer 401, and polymerization may be initiated to form a coating or shell around the first polymer 400.

In some alternative aspects, the second polymer 401 may be grown within the first polymer 400. In such aspects, the first polymer 400 may be synthesized with a porous or network structure to allow the penetration of the monomers of the second polymer 401. In such aspects, the monomers of the second polymer 401 may be infused into the porous structure of the first polymer 400. Exemplary schematics are shown in FIGS. 5A-C and 6A-C (showing individual first and second monomer structures, and resulting interpenetrating network structures). In such aspects, the infused monomers of the second polymer 401 may be polymerized within the structure of the first polymer 400 using techniques including, but not limited to, bulk polymerization or interfacial polymerization. In some aspects, the second polymer 401 may be formed in situ within the matrix of the first polymer 400 using, for example, polymerization or chemical transformation of the monomers.

In some alternative aspects, the second polymer 401 may be formed around the first polymer 400. In such aspects, the first polymer 400 may be synthesized and prepared as described above. In such aspects, monomers of the second polymer 401 may be deposited onto the surface of the first polymer 400. In such aspects, the monomers of the second polymer 401 on the surface may be polymerized or chemically transformed to form a continuous layer of second polymer 401 around the first polymer 400. In such aspects, the layers may be synthesized using layer-by-layer assembly, whereby alternating layers of monomer of the second polymer 401 are deposited and polymerized. In some aspects, the layers may be synthesized by sol-gel processing, whereby monomers of the second polymer 401 are processed to form a gel that subsequently forms a second polymer layer 401. In some aspects, a combination of these techniques may be used.

In some aspects, the first polymer 400 and second polymer 401 may be chemically linked. In some aspects, the first polymer 400 may be functionalized with reactive groups. In some aspects, the second polymer 401 may be functionalized with reactive groups. In some aspects, both the first polymer 400 and the second polymer 401 may be functionalized with reactive groups. In some aspects, the reactive groups of the first polymer 400 and the second polymer 401 may be brought together to form covalent bonds. In some aspects, the first polymer 400 and second polymer 401 may be chemically linked via covalent bonds. In some aspects, the chemical linkage may involve click chemistry, whereby azide-alkyne cycloaddition links the first polymer 400 to the second polymer 401. In some aspects, the chemical linkage may involve condensation reactions, whereby carboxyl and amine groups may form bonds which link the first polymer 400 and the second polymer 401.

In some aspects, the first and second polymers 400 and 401 may be physically linked. In some aspects, the first polymer 400 and second polymer 401 may be synthesized separately. In some aspects, the first polymer 400 and second polymer 401 may be physically blended. In some aspects, the first polymer 400 and second polymer 401 may be physically mixed. In some aspects, the first polymer 400 and second polymer 401 may be physically entangled. In some aspects, the first polymer 400 and second polymer 401 may be processed in a way such that their structures interlock physically. In some aspects, the first polymer 400 and second polymer 401 may be combined using melt blending, electrospinning, or co-extrusion.

### Exemplary Polymers

In some aspects, the invention may include one or more polymers. In some aspects, the one or more polymers may be hydrophilic. In some aspects, the one or more polymers may be crosslinked. In some aspects, the one or more polymers may be crosslinked to form a network structure.

In some aspects, the one or more polymers may be a hydrogel. In some aspects, the one or more polymers may be a hydrogel formed from hydroxyethyl methacrylate (HEMA). In some aspects, the one or more polymers may be a hydrogel formed from polyethylene glycol diacrylate (PEGDA). In some aspects, the one or more polymers may be a hydrogel formed from HEMA and PEGDA (HEMA/PEGDA).

In some aspects, the one or more polymers may be a formed from acrylamide-based polymers. In some aspects, the one or more polymers may be a hydrogel formed from polyacrylamide (PAM). In some aspects, the one or more polymers may be a hydrogel formed from N, N-methylenebis(acrylamide) (MBAA). In some aspects, the one or more polymers may be formed from natural polymers. In some aspects, the one or more polymers may be alginate. In some aspects, the one or more polymers may include calcium ions.

### Interpenetrating Networks

In some aspects, an interpenetrating network (or “interpenetrating polymer network,” or “IPN”) may be formed. In some aspects, the interpenetrating network may include two or more networks of polymers. It is understood that a “polymer” and a “polymer network” may be used interchangeably to describe an interconnected structure formed by the individual polymer chains, in some aspects within an interpenetrating network. In some aspects, the polymers within the interpenetrating network may be physically entangled. In some aspects, the polymers within the interpenetrating network may not be covalently bonded. In some aspects, the interpenetrating network may include a first polymer 400. In some aspects, the interpenetrating network may include a second polymer 401.

In some aspects, the interpenetrating network may include mechanical properties and stabilities beyond those of an individual polymer network. For example, in some aspects, the interpenetrating network may include a first polymer 400 and a second polymer 401. In some aspects, the first polymer 400, depending on its composition and based on its proposed utility, may provide benefits including, but not limited to, heightened strength, improved sensing elements, improved thermal stability, improved chemical resistance, improved electrical conductivity, high modulus, and/or abrasion resistance. In some aspects, the second polymer 401 may provide benefits including, but not limited to, biocompatibility, anti-oxidant properties, reduction of chemical degradation and/or oxidation, improved biodegradability, improved permeability, transparency, hydrophilicity, adhesion, and/or low toxicity. In some aspects, the interpenetrating network including the first polymer 400 and the second polymer 401 provides the combined properties of each polymer individually. In some aspects, the interpenetrating network may exhibit any combination of the properties of the first polymer 400 and the second polymer 401. In some aspects, the interpenetrating network may possess the properties of the first polymer 400, the properties of the second polymer 401, or a combination of the properties in any other matter. In some aspects, the interpenetrating network may offer a customizable platform where the selection and combination of first 400 and second 401 polymers may be tailored to achieve the desired properties, either individually or synergistically, to meet a specific use case.

An exemplary procedure for forming some aspects of an interpenetrating network is described below. In some aspects, an interpenetrating network may be formed by preparing a matrix of the first polymer 400. Exemplary formation(s) of polymers are described in the preceding section. In some aspects, the first polymer 400 of an interpenetrating network may be a hydrogel. In some aspects, the hydrogel may form porous architecture due to phase separation event(s) during a fabrication process. In some aspects, the second polymer 401 ma be added as described below to create an interpenetrating network or polymer architecture in the ‘blank’ or water phase of the first polymer 400.

In some aspects, monomers of a second polymer 401 may be introduced into the matrix of the first polymer 400 to create an interpenetrating network. In some aspects, the first polymer 400 may be soaked in a solution containing monomers of the second polymer 401. In some aspects, the first polymer 400 may be impregnated with monomers of the second polymer 401 under pressure. In some aspects, the first polymer 400 may be infiltrated by diffuse monomers of the second polymer 401. In some aspects, a surface of the first polymer 400 may be coated with a thin layer of monomers of the second polymer 401. Some alternative aspects may use different methods for introducing monomers of a second polymer 401 into a first monomer 400 to generate the interpenetrating network.

In some aspects, following the introduction of monomers of the first polymer 400 to the second polymer 401, a polymerization reaction may be initiated. In some aspects, the polymerization reaction may be activated by heat, UV radiation, the addition of a catalyst, or the addition of initiator molecules 405 as described throughout.

### Example 1

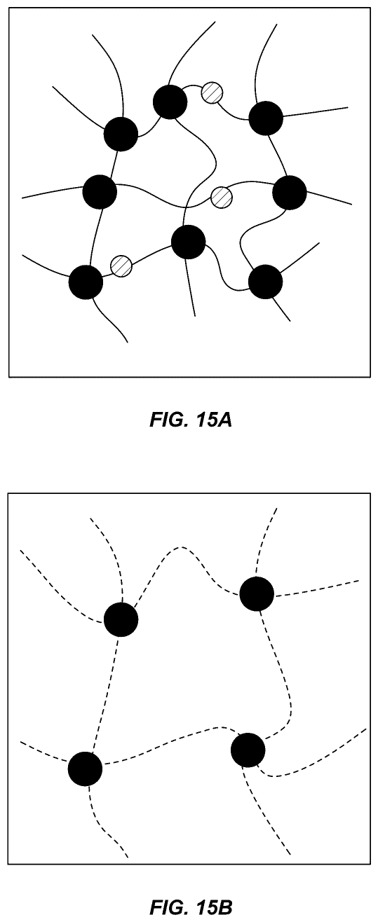

An interpenetrating network (IPN) including PEG-Thiol and PEG-Maleimide, as shown in FIGS. 15A-B was generated. As shown in FIG. 15A, the primary phase included a polymer-rich HEMA-PEGDA phase including an approximately 80% polymer volume fraction and low degrees of freedom. As shown in FIG. 15B, the secondary phase included a PEG Network-rich phase including approximately 10% polymer volume fraction.

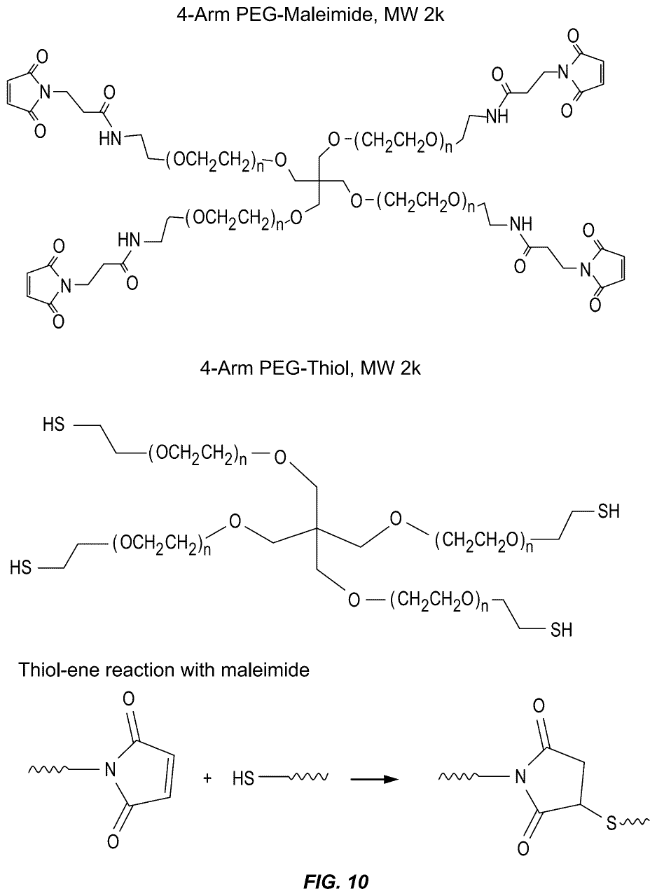

As shown in FIG. 10, the thiol group of the PEG-Thiol and the alkene group of the PEG-Maleimide underwent a Michael addition (thiol-ene reaction). The IPN was thus formed via step-growth polymerization using click chemistry, and did not require an initiator.

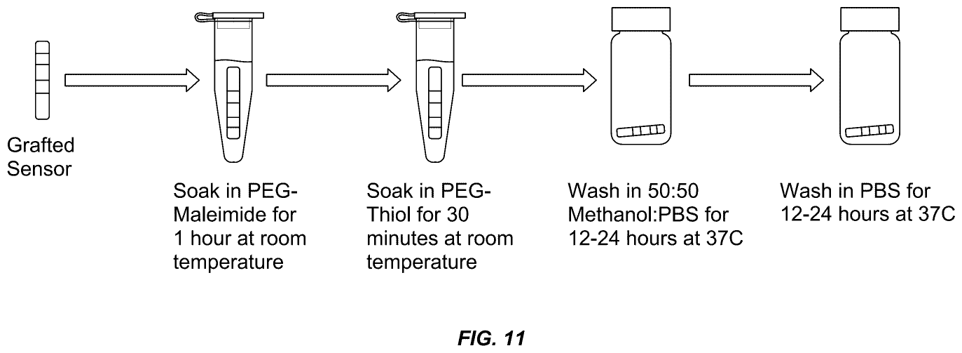

As shown in FIG. 11, the grafted sensor was first soaked in PEG-Maleimide at room temperature for 60 minutes. The sensor was then soaked in PEG-Thiol at room temperature for 30 minutes. The sensor was then washed in 50:50 methanol: PBS for 12-24 hours at 37° C. The sensor was then washed in PBS for 12-24 hours at 37° C.

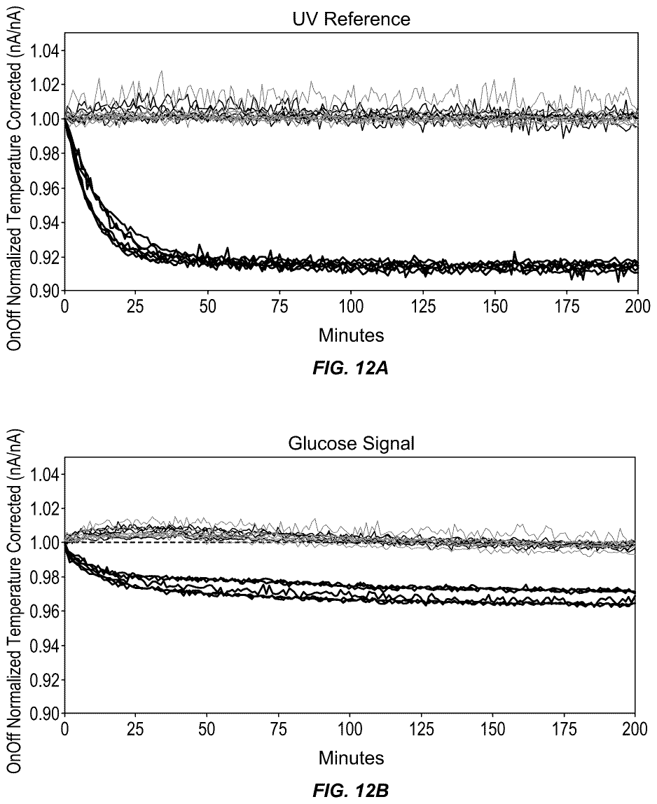

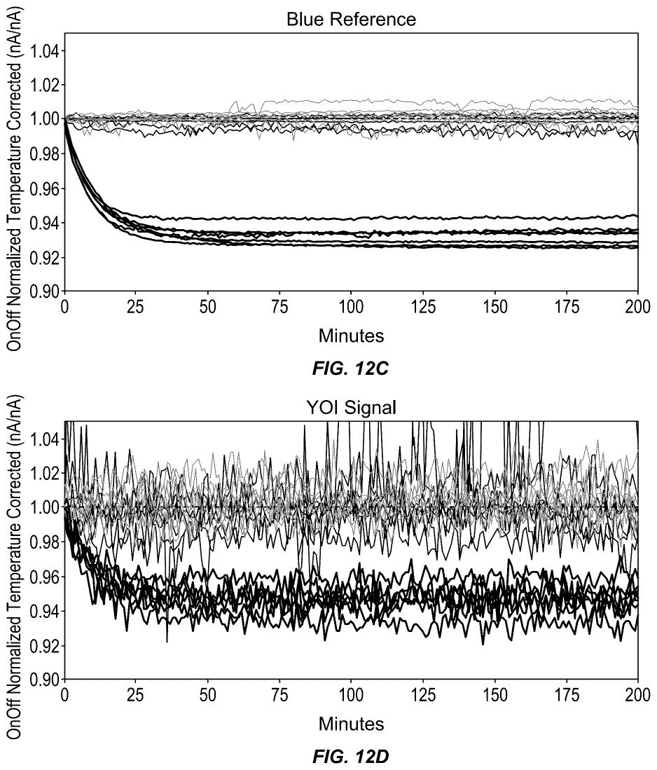

The sensor was tested for signal transmission in Bovine Serum Albumin (BSA). Results are shown in FIGS. 12A-D. As shown in FIGS. 12A-D, the sensor including the IPN at 10% (thin black line) and 7% (gray line) showed no change in any signal channel when exposed to BSA compared to a 3-9% decrease with a sensor without an IPN (thick black line).

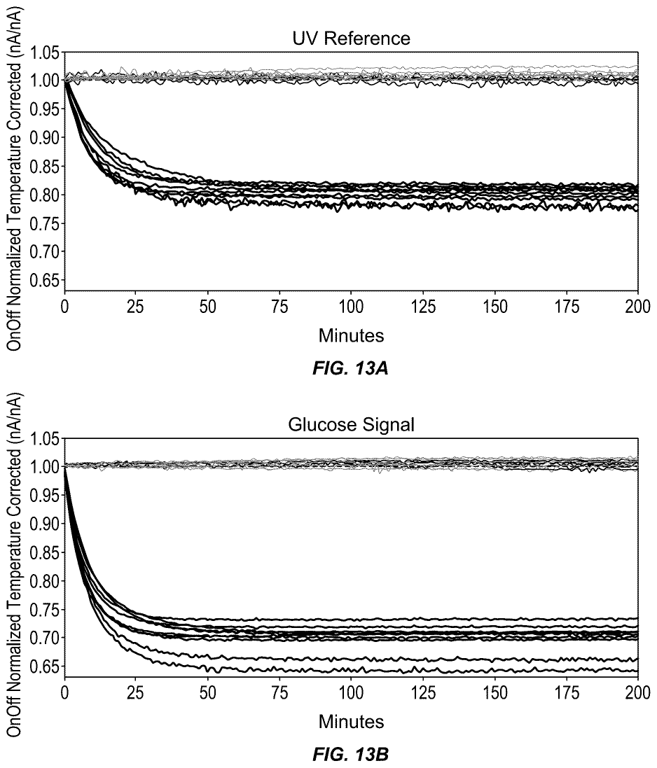

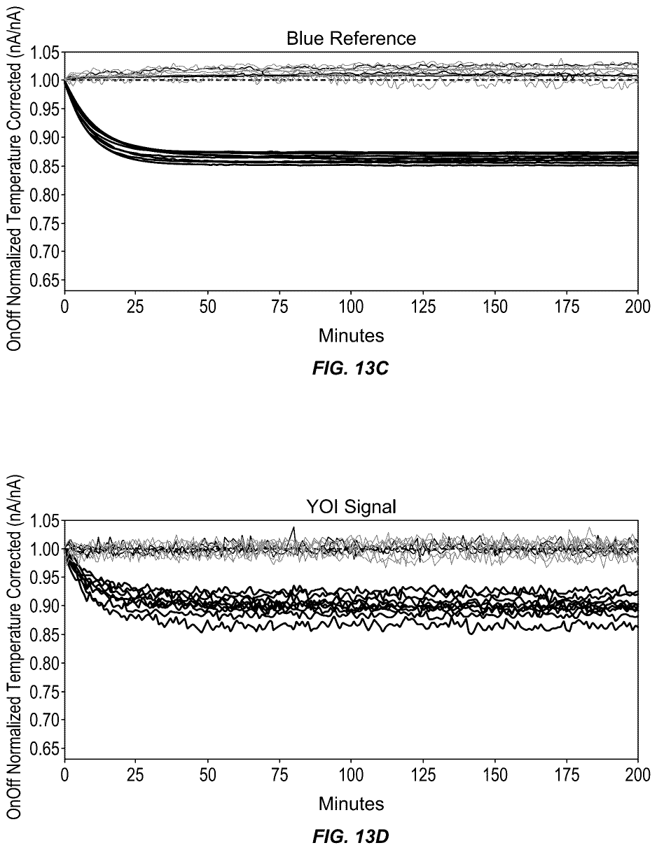

The sensor was tested for signal transmission in hemolyzed blood (hemoglobin). Results are shown in FIGS. 13A-D. As shown in FIGS. 13A-D, the sensor including the IPN at 10% (thin black line) and 7% (gray line) showed no change in any signal channel when exposed to BSA compared to a 10-30% decrease with a sensor without an IPN (thick black line).

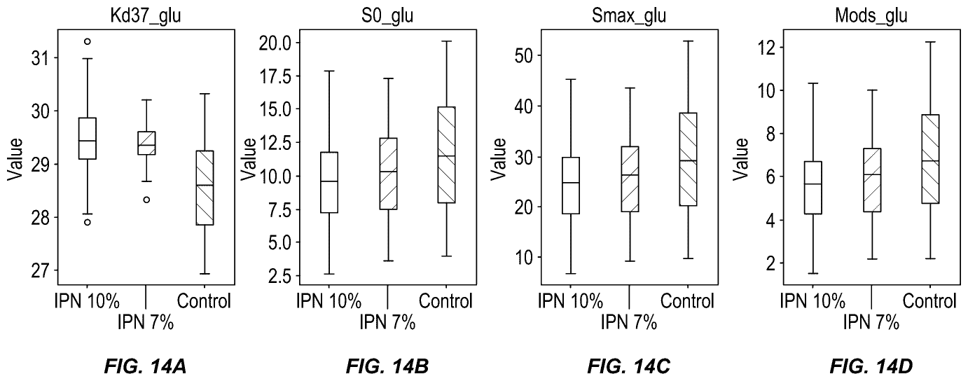

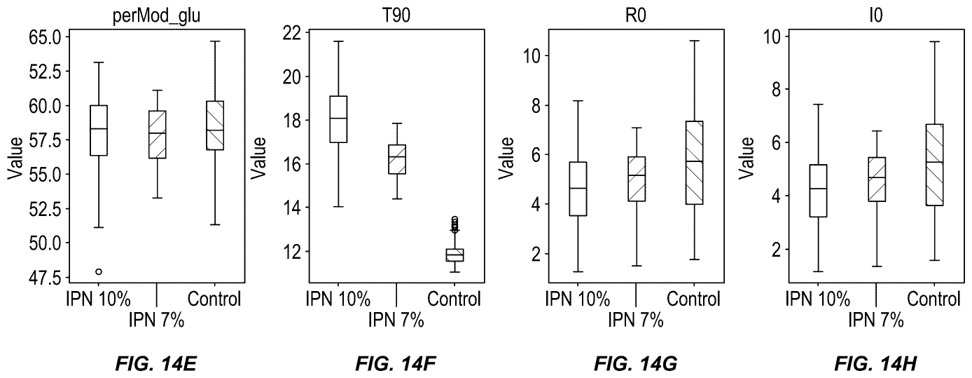

The sensor was tested post-graft for Sensor Functional Test (SFT) parameters as shown in FIGS. 14A-H. SFT parameters for the 10% IPN (left) and 7% IPN (middle) were within the MFS criteria for dissociation constant for glucose, Kd37_glu (FIG. 14A, baseline fluorescence intensity, S0_glu (FIG. 14B), maximum fluorescence intensity, Smax_glu (FIG. 14C), modulation of the sensor signal by glucose, Mods_glu (FIG. 14D), percentage modulation in the sensor signal with glucose, perMod_glu (FIG. 14E), baseline reference signal, R0 (FIG. 14G), and initial signal intensity, I0 (FIG. 14H). A six-minute increase with 10% IPN formulation was achieved. A four-minute increase with 7% IPN was achieved.

Thus, as shown in FIGS. 10-14, the generated IPNs were protein-resistant and biocompatible.

Aspects of the present invention have been fully described above with reference to the figures. Although the invention has been described based upon these preferred aspects, it would be apparent to those of skill in the art that certain modifications, variations, and alternative constructions could be made to the described aspects within the spirit and scope of the invention.

What is claimed is:

1. A sensor comprising:
a housing;
a first polymer; and
a second polymer;
wherein the first polymer comprises analyte indicator molecules and covers at least a portion of the housing; and
wherein the second polymer forms an interpenetrating network with the first polymer and reduces absorption and/or adsorption of one or more proteins, one or more macrophages, and/or one or more bacteria to the first polymer.

2. The sensor of claim 1, further comprising one or more light sources and one or more photodetectors within the housing.

3. The sensor of claim 1, wherein the second polymer is physically entangled with the first polymer.

4. The sensor of claim 1, wherein the second polymer further comprises analyte indicator molecules.

5. The sensor of claim 1, wherein the second polymer does not include analyte indicator molecules.

6. The sensor of claim 1, wherein the second polymer prevents optical interference of the sensor.

7. The sensor of claim 1, wherein the second polymer reduces contact of degradative species with the first polymer.

8. The sensor of claim 1, wherein the second polymer reduces, inhibits, or prevents electrostatic interactions of the first polymer with one or more proteins.

9. The sensor of claim 1, wherein the first polymer comprises co-monomers of four monomers according to Formula Ia: ABCD [Formula Ia],
wherein A is an analyte indicator monomer, B is a methacrylate monomer, C is a polyethylene glycol monomer, and D is a compound or monomer comprising boronate or boronic acid containing moieties.

10. The sensor of claim 1, wherein the second polymer comprises co-monomers of three monomers according to Formula Ib: EFG [Formula Ib],
wherein E is a methacrylate monomer, F is a polyethylene glycol monomer, and G is a compound or monomer comprising boronate or boronic acid containing moieties.

11. The sensor of claim 10, wherein the co-monomers of the second polymer are covalently linked via free radical polymerization, click chemistry or step growth polymerization.

12. The sensor of claim 10, wherein the F monomers of the second polymer are thiolene polymerized.

13. The sensor of claim 10, wherein the F monomers of the second polymer comprise N-hydroxysuccinimide (NHS), dibenzylcyclooctyne (DBCO), amine, epoxide, vinylsulfone, malemide, norebornene, thiol, azide, or alkyne groups.

14. A method of fabricating a sensor, the method comprising:
applying a first polymer to a housing of the sensor such that the applied first polymer covers at least a portion of the housing, wherein the first polymer comprises analyte indicator molecules;
swelling the first polymer;
soaking the first polymer in a solution comprising initiator molecules to form a soaked first polymer; and
placing the soaked first polymer into a monomer solution to form a second polymer that forms an interpenetrating network with the first polymer, wherein the second polymer reduces absorption and/or adsorption of one or more proteins, one or more macrophages, and/or one or more bacteria to the first polymer.

15. The method of claim 14, wherein the initiator molecules are selected from a group comprising free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amines, peroxide/ascorbic acid, ketone/amines), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

16. The method of claim 14, wherein the second polymer is chemically or physically linked to the first polymer.

17. The method of claim 16, wherein a solvent is used to control the extent to which the second polymer is physically linked to the first polymer.

18. The method of claim 16, wherein the duration that the first polymer is swelled is used to control the extent to which the second polymer is physically linked to the first polymer.

19. The method of claim 16, wherein the second polymer is physically entangled with the first polymer.

20. The method of claim 16, wherein the second polymer is grown from the first polymer.

21. The method of claim 14, wherein the second polymer further comprises analyte indicator molecules.

22. The method of claim 14, wherein the second polymer does not comprise analyte indicator molecules.

23. The method of claim 14, wherein the second polymer prevents optical interference of the sensor.

24. The method of claim 14, wherein the second polymer reduces contact of degradative species with the first polymer.

25. The method of claim 14, wherein the second polymer reduces, inhibits, or prevents electrostatic interactions of the first polymer with one or more proteins.

26. The method of claim 14, wherein the first polymer comprises co-monomers of four monomers according to Formula Ia: ABCD [Formula Ia], wherein A is an analyte indicator monomer, B is a methacrylate monomer, C is a polyethylene glycol monomer, and D is a compound or monomer comprising boronate or boronic acid containing moieties.

27. The method of claim 14, wherein the second polymer comprises co-monomers of three monomers according to Formula Ib: EFG [Formula Ib], wherein E is a methacrylate monomer, F is a polyethylene glycol monomer, and G is a compound or monomer comprising boronate or boronic acid containing moieties.

28. The method of claim 27, wherein the co-monomers of the second polymer are covalently linked via free radical polymerization, click chemistry or step growth polymerization.

29. The method of claim 27, wherein the F monomers of the second polymer are thiolene polymerized.

30. The method of claim 27, wherein the F monomers of the second polymer comprise N-hydroxysuccinimide (NHS), dibenzylcyclooctyne (DBCO), amine, epoxide, vinylsulfone, malemide, norebornene, thiol, azide, or alkyne groups.

31. The method of claim 14, further comprising drying the soaked first polymer before placing the soaked first polymer into the monomer solution to form the second polymer that covers at least the portion of the first polymer.

32. The method of claim 31, wherein the monomer solution comprises a mixture of initiator molecules and monomer molecules.

33. A method of fabricating a sensor, the method comprising:
immersing a housing in a solution comprising a first monomer, a second monomer, and initiator molecules; and
forming a first polymer and a second polymer that at least partially cover the housing, wherein the first polymer comprises analyte indicator molecules, and the second polymer forms an interpenetrating network with the first polymer.

34. The method of claim 33, wherein the initiator molecules are selected from a group comprising free radical polymerization initiators, including thermal initiators (including azo initiators, including azobisisobutyronitrile [AIBN], 1,1′-azobis(cyclohexanecarbonitrile) [ACHN] 2,2′-Azobis(2-methylbutyronitrile) [AMBN] and peroxide initiators, including di-tert-butyl peroxide (DTBP), dicumyl peroxide (DCP), benzoyl peroxide [BPO], dibenzoyl peroxide, and hydroperoxides, including tert-butyl peroxide [TBDP] and cumene hydroperoxide), photoinitiators (including benzoin ethers, benzil ketals, acetophenone derivatives, hydroxyalkylphenones, benzophenone derivatives, thioxanthone derivatives, camphorquinone, and anthraquinone derivatives), chemical (redox) initiators (including persulfate/bisulfite, hydrogen peroxide/Fe²⁺ [Fenton's reagent], permanganate/reducing agents, cerium(IV)/reducing agents, hydroperoxide/transition metals, peroxide/amines, peroxide/ascorbic acid, ketone/amines), cationic polymerization initiators (including Lewis acids, including aluminum chloride [AlCl₃] and boron trifluoride [BF₃], and protic acids, including sulfuric acid [H₂SO₄], and trifluoromethanesulfonic acid), anionic polymerization initiators (including organometallic compounds including n-butyllithium, sodium naphthalenide, and potassium amide [KNH₂]), and coordination polymerization initiators (including Ziegler-Natta catalysts, including titanium tetrachloride [TiCl₄], metallocene catalysts, including bis(cyclopentadienyl)titanium dichloride [Cp₂TiCl₂], and single-site catalysts, including nickel and palladium complexes).

35. A sensor comprising:
a housing;
a first polymer; and
a second polymer;
wherein the first polymer comprises analyte indicator molecules and covers at least a portion of the housing; and
wherein the second polymer forms an interpenetrating network with the first polymer and reduces chemical degradation and/or oxidation of the analyte indicator molecules.

36. A sensor comprising:
a housing;
a first polymer; and
a second polymer;
wherein the first polymer comprises analyte indicator molecules and covers at least a portion of the housing; and
wherein the second polymer forms an interpenetrating network with the first polymer and reduces chemical degradation and/or oxidation of the analyte indicator molecule and reduces absorption and/or adsorption of one or more proteins, one or more macrophages, and/or one or more bacteria to the first polymer.
